(19) United States
(12) Patent Application Publication
(10) Pub. No.: US 2026/0037099 A1
(43) Pub. Date: Feb. 5, 2026

(54) SPHERICAL ORB MEMBRANE ENVELOPE VISUALIZER GRAPHICAL USER INTERFACE SYSTEM, METHOD & COMPUTER PROGRAM PRODUCT

(71) Applicant: Methuselah Foundation, Springfield, VA (US)

(72) Inventor: David Gobel, Springfield, VA (US)

(73) Assignee: Methuselah Foundation, Springfield, VA (US)

(21) Appl. No.: 18/793,733

(22) Filed: Aug. 2, 2024

(51) Int. Cl. G06F 3/04815

(52) U.S. Cl. CPC G06F 3/04815

(57) ABSTRACT

A method, system or computer program product of electronically displaying a multi-dimensional complex data visualization graphical user interface (GUI) on an electronic display coupled to a computer processor and memory storage device, including: receiving sensed received data relating to an indicator; storing the sensed received data in the memory storage device; receiving the indicator detail of the indicator related to the sensed received data; and displaying on the GUI an avatar within a two dimensional rendering of a three dimensional orb visualizer of the sensed received data graphically displayed as a surface membrane including a data deformation include an intrusion data deformation or an extrusion data deformation of the surface membrane, where an envelope volume is formed between an inner surface of the surface membrane and an outer surface of the avatar, where the envelope is indicative of an associated relationship between the sensed received data and the indicator detail.

### CROSS REFERENCE TO RELATED APPLICATIONS

N/A

### BACKGROUND

### Field of the Disclosure

The present disclosure is generally related to computer software applications and more particularly to graphical user interfaces.

### Related Art

The present disclosure relates to graphical user interfaces. Early computers had simple output devices for displaying computer-based output, which initially provided monochromatic, initially text-based displays, allowing for a user to interact via a command line prompt such as, e.g. but not limited to, MICROSOFT's (of Redmond, WA USA) Disk Operating System (DOS) or IBM CORPORATION's (of Armonk, NY USA) PC-DOS, on the IBM personal computer (PC), the command line interface was interactively accessible by computer end users by using a keyboard or other input device, and using a series of batch language commands. Other output devices included computer printers. XEROX's (of Rochester, NY USA) Palo Alto Research PARC invented the first graphical user interface (GUI) an intuitive interface, later copied by APPLE COMPUTER of Cupertino, CA USA in the first MACINTOSH. IBM collaborated with Microsoft on a common user access (CUA) model for creating standardized window functions on its PCs, and eventually, later Microsoft created MICROSOFT WINDOWS versions 1.0, 2.0, 3.0, and eventually the widely popular version 3.1, and many later variations including, e.g., WINDOWS/NT, 1995, 1997, 8, 10, and its current version 11. The UNIX operating system originally from AT&T (of NJ USA), had its own early GUI, namely the X-Terminal Interface, available in many variations from AIX, HP/UX, to Linux. Other well known early GUIs included the NEXT Step OS developed when Steve Jobs left Apple, which later found itself in APPLE's MAC Operating System 10 (MACOSX), a UNIX based version of its operating system, still largely used on Apple's PCs today. Today's mobile device operating systems, primarily APPLE's iOS and Google's (from ALPHABET INC. of Mountain View, CA USA) ANDROID operating system (OS) (built largely on ORACLE's (of Austin, TX USA) JAVA acquired from SUN MICROSYSTEMS), also unlike well know GUIs.

GUIs thus provide intuitive ways for users to interact with data. Early improvements to data interaction include spreadsheets from VISICALC, to LOTUS 123, to QUATTRO PRO, to MICROSOFT OFFICE EXCEL and today to GOOGLE SHEETS, these provide two-dimensional matrix representations of numeric value and text-based data, and computer algebraic mathematical and logical operations, calculations with arithmetic functions and computations, allowing for simple graphing of data in two-dimensional charts. Conventional graphical visualizers have various shortcomings. The more complex data becomes, i.e., the greater the dimensions of data, the more difficult it becomes for users to grasp the information, using conventional visualization tools.

American mathematical statistician John Tukey, creator of the fast Fourier Transform (FFT) once wrote, “The greatest value of a picture is when it forces us to notice what we never expected to see.” We have all heard of the expression, “A picture is worth a thousand words.” Most conventional data visualizations today still show two-dimensional line and bar chart plots, many include histogram like bars representing data plotted along a two-dimensional set of axes, or a geometric Cartesian coordinate system of x/y plots of ordinate, and abscissa plots of a horizontal x axis and vertical y axis with an origin, and data points plotted thereon. Three dimensional data is often shown in an x/y/z, three-dimensional Cartesian coordinate systems of an ordered triplet of axes where each pair of axes defines a coordinate hyperplane and these hyperplanes break up into eight octants the system, often illustrated or displayed in volumetric multidimensional illustrations, bubble charts (where a size of bubble attempts to introduce a third dimension), or scatter plots. However, as data becomes more complex, visualization systems fail to be able to simply illustrate n-dimensional, real world data, where many more dimensions of metrics may exist than a mere two or three dimensions.

Conventional visualization tools fail to adequately meet market needs, and have various shortcomings. Conventional visualization tools fail to provide adequate functionality to address the needs of providing intuitive user friendly visual displays of complex multi-dimensional data in easy to use fashion. What is needed is an improved graphical user interface that overcomes the shortcomings of conventional apps.

### SUMMARY OF THE DISCLOSURE

According to an exemplary embodiment of the invention, the disclosure sets forth systems, methods and computer program products relating to visualization systems and methods and provides for various example embodiments.

According to one example embodiment, a computer-implemented method of electronically displaying a multi-dimensional complex data visualization in at least one graphical user interface (GUI) on at least one electronic display coupled to at least one electronic computer processor and at least one electronic memory storage device, the method may include: a) electronically receiving, by the at least one electronic computer processor, at least one sensed received data relating to at least one indicator; b) electronically storing, by the at least one computer processor, the at least one sensed received data in the at least one electronic memory storage device; c) electronically receiving, by the at least one computer processor, at least one indicator detail of the at least one indicator related to the at least one sensed received data; and d) electronically displaying in the graphical user interface, by the at least one computer processor, at least one avatar within an at least two dimensional rendering of an at least three dimensional orb visualizer the at least one sensed received data graphically displayed as at least one surface membrane may include at least one data deformation may include at least one of at least one intrusion data deformation or at least one extrusion data deformation of the at least one surface membrane, wherein an envelope volume is formed between an inner surface of the at least one surface membrane and an outer surface of the at least one avatar, wherein the envelope is indicative of an associated relationship between the at least one sensed received data and the at least one indicator detail.

According to one example embodiment, the method may include where the at least one graphical user interface (GUI) may include at least one or more of: an internet browser hypertext markup language (HTML) app; a messenger app; a texting app; an IOS-compatible app; an ANDROID-compatible app; a mobile OS-compatible app; an electronic messaging app; an electronic widget app; an hypertext transfer protocol (HTTP) browser-enabled app; an hypertext markup language (HTML) enabled app; a web-based application; a JAVA-compatible app; a PYTHON script app; or an interactive graphical user interface app.

According to one example embodiment, the method may include where the at least one graphical user interface (GUI) may further include at least one or more of: c) electronically illustrating in the graphical user interface, by the at least one computer processor, using at least one color level displayed in the GUI, associated with a severity level of the at least one sensed received data relative to the at least one indicator detail displayed graphically as the at least one surface membrane may include at least one intrusion data deformation, or at least one extrusion data deformation of the at least one surface membrane of the at least three dimensional orb visualizer; f) electronically representing visually by displaying, by the at least one computer processor, a severity of the at least one sensed data by the relative spatial proximity illustrated between the inner surface of the at least one surface membrane and an outer surface of the at least one avatar; g) electronically enabling zooming in to display, by the at least one computer processor, of the GUI of the at least three dimensional orb visualizer; h) electronically enabling interactive timeline review display, by the at least one computer processor, of the GUI of the at least three dimensional orb visualizer; i) electronically enabling interactive temporal increment of historical data display, by the at least one computer processor, of the GUI of the at least three dimensional orb visualizer; j) electronically enabling interactive filtering of severity indicator information data display, by the at least one computer processor, of the GUI of the at least three dimensional orb visualizer; or k) electronically enabling note capture and display, by the at least one computer processor, on the GUI of the at least three dimensional orb visualizer.

According to one example embodiment, the method may include where the at least one graphical user interface (GUI) may further include: c) electronically illustrating in the graphical user interface, by the at least one computer processor, using at least one color level displayed in the GUI, associated with a severity level of the at least one sensed received data relative to the at least one indicator detail displayed graphically as the at least one surface membrane may include at least one intrusion data deformation, or at least one extrusion data deformation of the at least one surface membrane of the at least three dimensional orb visualizer; f) electronically representing visually by displaying, by the at least one computer processor, a severity of the at least one sensed data by the relative spatial proximity illustrated between the inner surface of the at least one surface membrane and an outer surface of the at least one avatar; g) electronically enabling zooming in to display, by the at least one computer processor, of the GUI of the at least three dimensional orb visualizer; h) electronically enabling interactive timeline review display, by the at least one computer processor, of the GUI of the at least three dimensional orb visualizer; i) electronically enabling interactive temporal increment of historical data display, by the at least one computer processor, of the GUI of the at least three dimensional orb visualizer; j) electronically enabling interactive filtering of severity indicator information data display, by the at least one computer processor, of the GUI of the at least three dimensional orb visualizer; and k) electronically enabling note capture and display, by the at least one computer processor, on the GUI of the at least three dimensional orb visualizer.

According to one example embodiment, the method may include where the at least one sensed received data was captured at a time stamp and wherein the at least one sensed received data is associated with a user.

According to one example embodiment, the method may include where the user may include at least one or more of: a patient user; an entity; an asset; a vehicle; an automobile; an aircraft; a plane; a boat; an animal; a pet; a dog; a horse; a cat; or a cow.

According to one example embodiment, the method may include where the at least one avatar may include at least one or more of: an avatar of a patient user; an avatar of a female patient user; an avatar of a male patient user; a user customizable appearance avatar of a patient user; a user selectable avatar of a user; an avatar of an entity; an avatar of an asset; an avatar of a vehicle; an avatar of an automobile; an avatar of an aircraft; an avatar of a plane; an avatar of a boat; an avatar of an animal; an avatar of a pet; an avatar of a dog; an avatar of a horse; an avatar of a cat; or an avatar of a cow.

According to one example embodiment, the method may include where the at least one indicator detail may include at least one or more of: at least one biomarker indicator detail; at least one health test indicator detail; at least one health metric indicator detail; or at least one physical health related detail.

According to one example embodiment, the method may include where the at least one indicator detail may include at least one or more of: at least one range; at least one optimal range; at least one minimum; at least one maximum; at least one normal range; at least one normal minimum; at least one normal maximum; at least one importance; at least one category of the indicator; or at least one units of the indicator.

According to one example embodiment, the method may include where the at least one indicator detail may include at least one or more of: at least one asset maintenance related indicator detail; at least one asset test indicator detail; at least one asset maintenance metric indicator detail; or at least one asset service-related detail.

According to one example embodiment, the method may include where the at least three dimensional orb visualizer may include at least one or more of: a spherical orb shape; an elliptical orb shape; an amorphous continuous encapsulation shape; an egg orb shape; an oval orb shape; an enclosed orb shape; a cube orb shape; or a volumetric orb shape.

According to one example embodiment, the method may include where the at least one graphical user interface (GUI) may include at least one or more of: a software as a service (Saas) cloud-hosted application service provider software application; a natural language search query tool for interacting with the software application; a touchscreen pinch/zoom enabled application; a mouse pointer enable application; a generative artificial intelligence (AI) pre-trained machine learning (ML) large language model (LLM) natural language query tool for interacting with the software application; a deep learning convolutional neural network enabled generative artificial intelligence (AI) pre-trained machine learning (ML) large language model (LLM) natural language query tool for interacting with the software application; or a deep learning convolutional neural network enabled generative artificial intelligence (AI) pre-trained machine learning (ML) large language model (LLM) natural language query tool for interacting with the software application may include speech recognition and speech synthesis, natural language interaction with the software application.

According to another example embodiment, an example system of electronically displaying a multi-dimensional complex data visualization in at least one graphical user interface (GUI) application, the system may include: at least one electronic computer processor; at least one electronic touchscreen display input output device or at least one display and at least one mouse pointer device; at least one electronic memory storage device; and at least one operating system executing upon the at least one electronic computer processor the electronic computer processor configured to provide the at least one graphical user interface (GUI) application configured to electronically display the multi-dimensional complex data visualization, wherein the at least one electronic computer processor of the system is configured to: a) electronically receive at least one sensed received data relating to at least one indicator; b) electronically store the at least one sensed received data in the at least one electronic memory storage device; c) electronically receive at least one indicator detail of the at least one indicator related to the at least one sensed received data; and d) electronically display in the at least one graphical user interface at least one avatar within an at least two dimensional rendering of an at least three dimensional orb visualizer the at least one sensed received data graphically displayed as at least one surface membrane may include at least one data deformation may include at least one of at least one intrusion data deformation or at least one extrusion data deformation of the at least one surface membrane, wherein an envelope volume is formed between an inner surface of the at least one surface membrane and an outer surface of the at least one avatar, wherein the envelope is indicative of an associated relationship between the at least one sensed received data and the at least one indicator detail.

According to one example embodiment, the system may include where the at least one sensed received data may be captured from at least one electronic sensor on at least one or more of: at least one sensor-based device; at least one wearable device; at least one health sensor-based device; at least one physical health information capture device; or at least one mobile device may include at least one sensor.

According to yet another example embodiment, a non-transitory computer program product embodied on a computer accessible storage medium, may include at least one instruction, which when executed on at least one electronic computer processor may perform a method of electronically displaying a multi-dimensional complex data visualization in at least one graphical user interface (GUI) on at least one electronic display coupled to at least one electronic computer processor and at least one electronic memory storage device, the method may include: a) electronically receiving, by the at least one electronic computer processor, at least one sensed received data relating to at least one indicator; b) electronically storing, by the at least one computer processor, the at least one sensed received data in the at least one electronic memory storage device; c) electronically receiving, by the at least one computer processor, at least one indicator detail of the at least one indicator related to the at least one sensed received data; and d) electronically displaying in the graphical user interface, by the at least one computer processor, at least one avatar within an at least two dimensional rendering of an at least three dimensional orb visualizer the at least one sensed received data graphically displayed as at least one surface membrane may include at least one data deformation may include at least one of at least one intrusion data deformation or at least one extrusion data deformation of the at least one surface membrane, wherein an envelope volume is formed between an inner surface of the at least one surface membrane and an outer surface of the at least one avatar, wherein the envelope is indicative of an associated relationship between the at least one sensed received data and the at least one indicator detail.

According to one example embodiment, the method may further include, e.g., but not limited to, one or more of: f) electronically receiving, by the at least one electronic computer processor, a request to share and electronically sharing access to the graphical user interface to an authorized third party via at least one or more of: electronic social network; electronic communication; electronic mail; electronic message; electronic social media post; or electronic access control; g) electronically gamifying, by the at least one electronic computer processor, receiving a request to enable share to a plurality of users and electronically sharing access to the graphical user interface to said plurality of users, authorized to have access to the GUI via at least one or more of: electronic social network; electronic communication; electronic mail; electronic message; electronic social media post; or electronic access control; or h) electronically receiving, by the at least one electronic computer processor, a request to share and electronically sharing access to the graphical user interface to an authorized third party wherein the authorized third party comprises least one or more of: a physician; a service provider; a doctor; an insurance carrier; a care giver; a health care provider; a family member; a group of users; a group of competitors in a competition; an owner of an entity; an owner of an asset; or an artificial intelligence/machine learning deep learning analyzer.

According to one example embodiment, the method may further include, e.g., but not limited to, one or more of: f) electronically receiving, by the at least one electronic computer processor, a request to electronically customize said avatar and electronically providing user access via the graphical user interface to tools to customize an appearance of said avatar comprising at least one or more of: electronically selecting an example gender customized appearance of said avatar; electronically selecting an example realistically featured customized appearance of said avatar; electronically selecting an example single color appearance of said avatar; electronically customizing an appearance of said avatar via artificial intelligence and/or machine learning; electronically customizing an appearance of said avatar via user and/or automated selection of a selection from a library of example avatar models; electronically customizing an appearance of said avatar comprising at least one or more of: selecting an approximately similarly aged avatar; selecting an animated avatar; selecting a caricatured avatar; selecting a realistically simulated avatar; selecting a 3D scanned avatar; selecting an automatically changed avatar; selecting a posed avatar; selecting a seasonally clothed avatar; selecting a transparent avatar; or selecting a group of a plurality of avatars; or g) electronically receiving, by the at least one electronic computer processor, a request to electronically perform a what if scenario, and automatically customizing said avatar and electronically displaying via the graphical user interface said customized appearance of said avatar comprising at least one or more of: electronically customizing based on performing at least one extrapolation of a trend of past historical data, into a predictive future based on said what if scenario to at least one or more of: display an improvement in appearance of said avatar if said at least one extrapolation based on said what if scenario is desirable; display a change in appearance of said avatar; display an undesirable change in appearance of said avatar if said at least one extrapolation is undesirable; display a change in appearance of said avatar based on an artificial intelligence or machine learning neural network of expected changes based on said what if scenario; display a change in appearance of at least a portion of said avatar based on an artificial intelligence or machine learning neural network of expected changes based on said what if scenario; or display a change in appearance of at least a portion of at least one internal organ of said avatar based on a predicted outcome of said what if scenario.

According to one example embodiment, the method may further include, e.g., but not limited to, one or more of: f) electronically receiving, by the at least one electronic computer processor, a request to share and electronically sharing access to the graphical user interface to an authorized third party via at least one or more of: electronic social network; electronic communication; electronic mail; electronic message; electronic social media post; or electronic access control; g) electronically gamifying, by the at least one electronic computer processor, receiving a request to enable share to a plurality of users and electronically sharing access to the graphical user interface to said plurality of users, authorized to have access to the GUI via at least one or more of: electronic social network; electronic communication; electronic mail; electronic message; electronic social media post; or electronic access control; h) electronically receiving, by the at least one electronic computer processor, a request to share and electronically sharing access to the graphical user interface to an authorized third party wherein the authorized third party comprises least one or more of: a physician; a service provider; a doctor; an insurance carrier; a care giver; a health care provider; a family member; a group of users; a group of competitors in a competition; an owner of an entity; an owner of an asset; or an artificial intelligence/machine learning deep learning analyzer; i) electronically receiving, by the at least one electronic computer processor, a request to electronically customize said avatar and electronically providing user access via the graphical user interface to tools to customize an appearance of said avatar comprising at least one or more of: electronically selecting an example gender customized appearance of said avatar; electronically selecting an example realistically featured customized appearance of said avatar; electronically selecting an example single color appearance of said avatar; electronically customizing an appearance of said avatar via artificial intelligence and/or machine learning; electronically customizing an appearance of said avatar via user and/or automated selection of a selection from a library of example avatar models; electronically customizing an appearance of said avatar comprising at least one or more of: selecting an approximately similarly aged avatar; selecting an animated avatar; selecting a caricatured avatar; selecting a realistically simulated avatar; selecting a 3D scanned avatar; selecting an automatically changed avatar; selecting a posed avatar; selecting a seasonally clothed avatar; selecting a transparent avatar; or selecting a group of a plurality of avatars; and j) electronically receiving, by the at least one electronic computer processor, a request to electronically perform a what if scenario, and automatically customizing said avatar and electronically displaying via the graphical user interface said customized appearance of said avatar comprising at least one or more of: electronically customizing based on performing at least one extrapolation of a trend of past historical data, into a predictive future based on said what if scenario to at least one or more of: display an improvement in appearance of said avatar if said at least one extrapolation based on said what if scenario is desirable; display a change in appearance of said avatar; display an undesirable change in appearance of said avatar if said at least one extrapolation is undesirable; display a change in appearance of said avatar based on an artificial intelligence or machine learning neural network of expected changes based on said what if scenario; display a change in appearance of at least a portion of said avatar based on an artificial intelligence or machine learning neural network of expected changes based on said what if scenario; or display a change in appearance of at least a portion of at least one internal organ of said avatar based on a predicted outcome of said what if scenario.

According to one example embodiment, the method may include where the (b) of said electronically storing comprises at least one or more of: electronically storing said at least one sensed received data in at least one hierarchical database; electronically storing said at least one sensed received data in at least one relational database; electronically storing said at least one sensed received data in at least one structured query language (SQL) relational database; electronically storing said at least one sensed received data in at least one graph database; or electronically storing said at least one sensed received data in at least one No SQL graph database.

According to one example embodiment, the method may include, e.g., but not limited to, where the (d) of said electronically displaying in the graphical user database comprises electronically displaying at least one predictive change in appearance to said avatar, based on at least one what if scenario.

### BRIEF DESCRIPTION OF THE DRAWINGS

The subject matter herein is particularly pointed out and is distinctly claimed in the claims at the conclusion of the specification. The foregoing and other objects, features and advantages will be apparent from the following detailed description taken in conjunction with the accompanying drawings.

The accompanying drawings, which are included to provide a further understanding and are incorporated herein and constitute a part of this specification, illustrate exemplary, illustrative and nonlimiting embodiments and together with their description serve to explain the principles disclosed herein. In the drawings, like reference numbers may indicate substantially similar, equivalent, or exemplary elements, and the left most digit(s) in the corresponding reference number can indicate the drawing in which an element first appears.

FIG. 1 depicts an example embodiment of an example screen capture of an example spherical orb graphical user interface visualizer in an example light mode view of an illustrative example power user interface including an example uses avatar illustrated within, an example horizontal axis slider GUI element timeline, an example left vertical axis slider GUI element for varying time increments, and example right vertical axis slider GUI element for varying/filtering amount of severity data displayed in the visualizer, according to an example embodiment of the present invention;

FIG. 2 depicts an example embodiment of an example screen capture of an example spherical orb graphical user interface visualizer in an example dark mode view of an illustrative severity ranges view illustrating example membrane example deformations of example indicator intrusions and extrusions and forming an example volumetric envelope between the example membrane surface and including an example user avatar illustrated within, and example zoomed in example view of a zoomed in spherical orb visualizer UI, according to an example embodiment of the present invention;

FIG. 3 depicts an example screen capture of an example on-screen ‘How to Use’ tutorial displaying various help information, according to example embodiments of the present invention;

FIG. 4A depicts an example embodiment of an example viewmaster selection menu including example favorite star elements and multiple numeric access counters and dark and light view, severity and hazard range view as well as example light mode severity views with example high, medium and low severity indicators for example sensed data graphically displayed on an example spherical orb graphical user interface, according one example embodiment of the present invention;

FIG. 4B depicts an example embodiment of an example viewmaster selection GUI button of visualizer including example female gender avatar displayed on an example spherical orb graphical user interface, according one example embodiment of the present invention;

FIG. 5 depicts an example embodiment of an example dark mode view of an example spherical orb membrane user interface with example indicator intrusions and extrusions illustrating using example color-based severity example indications, and defining an amorphous three dimensional envelope volume between the example inner surface of the membrane and the illustrated example user avatar and illustrating by proximity example health data with detrimental sensed data appearing proximate in distance with example intrusions coming near to the avatar, and including a timeline GUI element for browsing backward or forward through various timestamped sensed data, according to one example embodiment of the present invention;

FIG. 6A depicts another example embodiment of an example dark mode view of an example spherical orb membrane user interface with example indicator intrusions and extrusions illustrating using example color-based severity example indications, additional severity level variation illustrations, example outer membrane extrusion positive indicators, and defining the amorphous three dimensional envelope volume between the example inner surface of the membrane and the illustrated example user avatar and illustrating by proximity example health data with detrimental sensed data appearing proximate in distance with example membrane intrusion indicators coming near to the avatar, and including a timeline GUI element for browsing backward or forward through various timestamped sensed data, example play/pause button, example jump to first data, example jump to last data, example indicator scroll bars, additional details, diagnoses section, medication section, visualizer and indicator list including severity color-based indicators and numeric severity metric values, and example note area, example starry background in example view, according to one example embodiment of the present invention;

FIG. 6B depicts another example embodiment of an example dark mode view of an example spherical orb membrane user interface illustrating the example horizontal and vertical sliders of the example visualizer and example note adding popup data entry window, according to one example embodiment of the present invention;

FIG. 7 depicts an example embodiment of an indicator addition/editing/enabling/disabling menu, according to one example embodiment of the present invention;

FIG. 8 depicts an example embodiment of an indicator sorting options pulldown menu, according to one example embodiment of the present invention;

FIG. 9 depicts an example embodiment of an example screen capture of an example edit a given example cholesterol indicator with example indicator biomarker details example fields popup window, according to an example embodiment of the present invention;

FIG. 10 depicts an example embodiment of an example schematic diagram of an example illustration of an example computer hardware architecture block diagram of an example component of any of various example devices of an example system, according to one example embodiment of the present invention;

FIG. 11 depicts an example embodiment of an example schematic diagram of an example illustration of an example computer load balancing example system architecture block diagram of various example system components of any example networked system of various example electronic computing devices of an example system, according to one example embodiment of the present invention; and

FIG. 12 depicts an example embodiment of an example flow chart diagram illustrating an example high level flow chart of example process flow of an example display visualizer user interface apparatus, method, system and computer program product, according to one example embodiment of the present invention.

### DETAILED DESCRIPTION OF VARIOUS EXEMPLARY EMBODIMENTS

It is important to note that the embodiments disclosed are only examples of the many advantageous uses of the innovative teachings herein. In general, statements made in the specification of the present application do not necessarily limit any of the various claims. Moreover, some statements may apply to some inventive features but not to others. In general, unless otherwise indicated, singular elements may be in plural and vice versa with no loss of generality. In the drawings, like reference numerals may refer to like parts through several views.

Reference will now be made in detail to various exemplary embodiments, examples of which are illustrated in the accompanying drawings. While specific exemplary embodiments are discussed, it should be understood that this is done for illustration purposes only. A person skilled in the relevant art will recognize that other components and configurations may be used without parting from the spirit and scope of the disclosure.

The following detailed description is of the best currently contemplated modes of carrying out various exemplary embodiments of the invention. The description is not to be taken in a limiting sense, but is made merely for the purpose of illustrating the general principles of the present invention.

### Overview of an Exemplary Embodiment of an Example Spherical Orb Membrane Envelope Visualizer Graphical User Interface (GUI) Application

Broadly, an example embodiment of the present invention may provide an example computer application, which may include, e.g., but not limited to, a web-based application, an example hypertext markup language (HTML) based web-browser based application, an applet, a JAVA based applet and/or application, a local and/or remote and/or hybrid client/server based software application executing on a personal computer operating system, and/or or a mobile application based application such as, e.g., but not limited to, an application executing on a mobile operating system and/or an iOS and/or Android operating system application and/or a related software as a service cloud-based and/or application server based system and method for enabling users to access an example visualizer application graphical user interface, according to various example embodiments.

In an exemplary embodiment, the system, method or computer program product may include, e.g., but not limited to, at least one exemplary electronic computer and/or mobile device with, e.g., but not limited to, an exemplary computer processor, and an exemplary user interface such as, e.g., but not limited, a graphical user interface (GUI), etc., according to an exemplary embodiment. The exemplary computer may include at least one electronic computer processing unit coupled to at least one form of memory device, according to an exemplary embodiment. The exemplary computer may include, e.g., but not limited to, an example microprocessor with at least one central processing unit and/or a system on a chip and/or other microcontroller built with any of various conventional architectures such as, e.g., but not limited to, an ARM architecture, an INTEL architecture, an AMD architecture, etc. The exemplary computer may include, e.g., but not limited to, a server, a desktop, a laptop, a communications device, a client, a thin client, a mobile device, a smart device, such as, a tablet and smart phone, etc., according to an exemplary embodiment. The exemplary computer may include a computer program product including a machine-readable program code for causing, when executed, the computer to perform exemplary steps of an exemplary method, according to an exemplary embodiment. The exemplary computer program product may include, e.g., but not limited to, exemplary software instructions, which may either be loaded onto the computer or accessed by the computer, and may be executable on the exemplary computer's processor. The loaded software instructions may include an application program or app, downloaded onto a smart device or mobile device, such as, e.g., but not limited to, a smartphone to be executed on the mobile device's processor to perform an exemplary method, according to an exemplary embodiment. The software application program may alternatively be an application which may be executed on a computing device, such as, e.g., but not limited to, natively on a computer processor, or accessed by the computer using a web browser to interact with one or more hyperlinked computing devices on an exemplary world wide web (WWW) of the global internet, according to an exemplary embodiment. The computer may access the software via the web browser using a proprietary network, the global internet, an internet, extranet, intranet, host server, application server, web server, SaaS, internet cloud and/or the like, etc., according to an exemplary embodiment.

The ordered combination of various ad hoc and automated tasks in the presently disclosed platform necessarily achieve technological improvements through the specific processes described in more detail below, according to an exemplary embodiment. In addition, the unconventional and unique aspects of these specific automation processes, according to an exemplary embodiment, represent a sharp contrast to merely providing a well-known or routine environment for performing a manual or mental task.

In one example aspect, an exemplary embodiment of the claimed invention may be an exemplary GUI visualizer application, which may be browser-based and/or local PC OS and/or remote application server based, and/or mobile application based, (such as, e.g., but not limited to, an exemplary iOS/Android application, etc.) that may serve as an exemplary system for users to interactively access and visualize complex multidimensional data about various example indicator metrics and to graphically display in the GUI visualizations of the complex data to provide intuitive information by illustrating in the visualization proximity of detrimental data to an avatar indicating the example severity of the detrimental data as compared to optimal and/or normal expected ranges for the sensed data, which is further timestamped and configured for allowing interactive user access to the historical changes to the sensed data displayed in the visualizer and enhanced with intuitive user selectable indicator details for user access and discussion with service providers such as physicians with respect to patient health care record data, according to one example, but nonlimiting embodiment. Various example embodiments include artificial intelligence/machine learning based interaction features including, e.g., but not limited to, example AI/ML enabled large language model (LLM) enabled natural language search queries and/or textual and/or voice interactive queries, natural language processing (NLP) user interaction to enable electronically interacting with example, other users, electronically communicating, electronically querying and viewing electronically displayed visualized data and enhanced with intuitive slider temporal horizontal axis sliders and play/pause, jump to first data, jump to last data, and vertical slider based temporal increment variation and/or filtering of severity data and/or level of hazard data, electronic communications, electronic health record data access, visualization of electronic plotting of complex actual sensed/captured data vs. optimal range and/or normal range, and/or dangerous range and/or severity level of example indicators, and/or electronic note taking and/or adding indicators and/or editing and/or exporting and/or importing indicators and/or groups of indicators, according to various example embodiments, various dark modes, various light modes, severity views, hazard views, various details views, information about service providers, information about users, information about patient users, information about physician users, information about next scheduled patient user appointments, information about medicine, information about medical interventions, information collection, capture, sensing, storage, data analysis, data tracking, intuitive, easy to use, GUI element interactions, slider elements, timeline elements, back buttons, x to return buttons, radio buttons, play/pause, jump to front, jump to back buttons, search fields, search query fields, note taking fields, note saving and/or access to notes, application help, application training, and enhanced example features, according to various example, but nonlimiting example embodiments. The example embodiments may include various other exemplary alternative embodiments.

### Example Embodiment of an Example MORRB: A Dynamic Representation of Example Biometric Data—Methuselah Foundation

### Introduction

According to one example embodiment, at a core of any preventive healthcare model is a powerful software system capable of making accurate, precise, and personalized inferences based on complex patient data, according to one example embodiment. According to one example embodiment, an example system of the disclosure is put forward as such an example system, according to one example embodiment.

First, an outline may be provided of an intuitive and novel data-driven framework for representing physiological states and predicting their evolution, according to one example embodiment. Then, an example software implementation of an example embodiment of this framework is described, according to one example embodiment. The central object of the application, according to one example embodiment, may be to provide, e.g., but not limited to, an example personalized bubble or sphere, according to one example embodiment, which may be termed and/or referred to herein as, MORRB, whose example surface topology may represent an example depicted example depiction of an example physical condition of the user, at a given point in time, associated with certain captured data or analyzed information of example data, according to one example embodiment. Every MORRB may be connected to and/or coupled to, e.g., but not limited to, several example high-quality biometric and/or example physiologic data streams particular to the example user, according to one example embodiment. The user may have an avatar depicting the user in the application, according to one example embodiment. An example avatar may be shown and/or displayed floating in, e.g., but not limited to, the middle of the MORRB, according to one example embodiment. Various example regions of the example bubble may be dedicated to the different aspects of health of the user (e.g., but not limited to, cardiovascular health, digestive health, psychological health, etc.), and an example decline determined by electronic analysis and/or comparison to example optimal and/or normal and/or abnormal ranges using e.g., normalized, consistent unit based sensed measures, in any of the various example multiple dimensions of example well-being may be visualized as an example depression in the surface and/or other deformation to the example bubble and/or membrane as otherwise described herein, and/or at an example corresponding region, etc., according to one example embodiment. The example depressions, according to one example embodiment, may cause protrusion within the bubble that may be, e.g., but not limited to, displayed, according to one example embodiment, as oriented inwardly towards the example illustration of the avatar in the example center location, according to one example embodiment. The example depth of the example depression at a particular example location and/or, equivalently, the example proximity of the example protrusion to the avatar, may, e.g., represent an example severity of the issue being brought to the attention of the user, according to one example embodiment. The goal of the user, according to one example embodiment, may then be to, e.g., but not limited to, take action, e.g., to cause change in the user's behavior so as to positively vary sensed data so as to, e.g., but not limited to, push back any example undesirable example protrusions threatening the example user avatar, according to one example embodiment. If the user, according to one example embodiment, may excel significantly in a particular aspect of improving the example user's health, the corresponding region may be electronically displayed as, e.g., but not limited to, expanding and/or jutting outwards from the example surface membrane of the example orb and/or bubble, according to one example embodiment.

First, according to one example embodiment, this disclosure shall focus on the example MORRB according to one example embodiment and may, e.g., but not limited to, may closely describe, e.g., in abstract example terms, the example orb GUI application's example input data, internal structure and/or example software algorithms, which may governing the example application's example evolution over time, according to one example embodiment. See, e.g., section entitled The MORRB under Input Data, Structure, and Dynamics.

Second, according to one example embodiment, may be detailed an example concrete software implementation of an example embodiment of the example MORRB in a section entitled The Software, according to one example embodiment. Here, may be enumerated various example functionalities and/or features of an example embodiment of the application (referred to herein as functionalities and/or features, which may be considered as example in nature, but nonlimiting in the application to the greater broader claimed invention), and as set forth herein in example detail may outline an example high-level architecture (Architecture), may describe the adapter bringing data in from example electronic medical records (EMR), personal health records (PHR), digitized and/or digitally captured, and/or electronically sensed, and/or electronically captured data and/or Electronic Medical Record (EMR), and/or accessed from example electronic adapters and/or sensors and/or sensing devices, and/or may outline, e.g., but not limited to, example wearable devices this application may connects to and/or couple to, in certain example embodiments, and the pieces of middleware that may facilitate this example connection and/or coupling (e.g., but not limited to, Wearables and Middleware, etc.), according to one example embodiment. Near the end of the example software section, according to one example embodiment, may be discussed example structure and/or example functions of an example database, which may serve as a basis of, and/or sit at an example heart of, the example application (e.g., but not limited to, Database, etc.) and may turn attention to example platform-specific implementation example details (such as, e.g., but not limited to, e.g., an example Web Platform, an example Mobile Platform, and/or an example VR Platform, etc.), according to one example embodiment.

### Example Embodiment of the MORRB

### Overview

In this section, according to one example embodiment, one may consider, in example abstract terms, an example structure of an example MORRB and an example mechanism and/or example input data, which may govern the example MORRB in its evolution of depiction, over time, according to one example embodiment. The example input data may be, e.g., but not limited to, e.g., sourced from EMRs, sensed from example biometric sensors and/or other example sensors on example wearable devices, etc., and/or, in some cases, the example data may be captured from prompted user interactive data entry via example fields prompting entry by user of example data, and receiving by the system that information and storing and analyzing the data for later access and/or analysis and electronically displayed interactive access along the lines of various example illustrative depicted displayed information, according to one example embodiment. The structure of the MORRB, according to one example embodiment, may be hierarchical, according to one example embodiment. According to other example embodiments, the structure may be, e.g., relational and may be stored in an example relational database, in another example embodiment, the data may be stored in example flat file format, and/or in another example embodiment may be accessible using example structured query language (SQL) example database access methods, and/or in another example embodiment may be stored in a No SQL database format, may be placed in an example graph database using an example map like graph to enable traversal of the example graph database with data captured in example nodes and interconnections between nodes, and/or via any of various other well known example database management systems (DBMSs) and/or other data storage and/or retrieval methodologies and may incorporate any of various example well know access control access methods such as, e.g., but not limited to, example public key encryption (Public/Private key) systems such as, e.g., but not limited to, example PKI systems and/or other access methods, authentication systems, password based authentication, two factor authentication (2 fa), and/or biometric access control systems, accessible using example systems, and displayed and provided for interactive user access via example input/output devices, mouse pointers, touch screen display interfaces, and/or other appropriate and useful system, as will be apparent to those skilled in the relevant art, according to one example embodiment. As for an example evolution, according to one example embodiment of the MORRB can be conceptualized as an example simple state machine, such as, e.g., but not limited to, an example finite state machine (FSM), which, as will be apparent to those skilled in the relevant art, may include a finite-state machine (FSM) or finite-state automaton (FSA, plural: automata), finite automaton, or simply a state machine, is may include a mathematical model of computation, according to one example embodiment. According to one example embodiment, an FSM or FSA may include an example abstract machine that can be in, according to one example embodiment, exactly one of a finite number of states at any given time, according to one example embodiment. Let S be the example current state of the MORRB as represented by its surface topology, let D be newly sourced input data representing an updated picture of the health of the user, and let T be the state transition function, according to one example embodiment. Periodically, according to one example embodiment, the example MORRB may integrate this new information, according to one example embodiment, by activating T, which can be defined as T:S×D→S′ where S′ is the updated state, according to one example embodiment.

### Example Input Data

According to one example embodiment, example Clinical lab tables may be sourced from EMRs and biometric information, and may include, according to one example embodiment, e.g., but not limited to, exercise data (e.g., but not limited to, type, duration, calories burned, etc.), sleep data (e.g., but not limited to, time spent awake and in each sleep cycle i.e. e.g., but not limited to, REM, Core, and Deep, and respiratory rate, etc.), oxygen saturation trends, cardiac data (i.e., e.g., but not limited to, heart rate trends and V02 max, etc.), ECG readings (if any), body temperature data, walking data (i.e., e.g., but not limited to, number of steps, double support time, walking asymmetry, walking step length, six-minute walk, walking steadiness, etc.), may be brought in by the application for analysis on a daily basis, according to one example embodiment. The entire medical history of the user, including information such as e.g., but not limited to, genetic predispositions, current symptoms, medications, and recent illnesses and operations or procedures may be taken into account and/or may be recorded from these sources of data, according to one example embodiment. Other data forming the backdrop of the clinical picture, such as, e.g., but not limited to, those regarding diet, psychological state, unhealthy habits, or social well-being, etc., may be obtained directly from the user, according to one example embodiment.

### Structure

According to one example embodiment, the example MORRB may be broken up into various example discrete regions, according to one example embodiment. Each region, according to one example embodiment, may correspond to a specific physiological system, according to one example embodiment. The following systems, according to one example embodiment, ma include and be represented: e.g., but not limited to, the endocrine system, immune system, reproductive system, central nervous system, peripheral nervous system, respiratory system, circulatory system, gastrointestinal system, renal system, and musculoskeletal system, etc. [NOTE: Determine precise arrangement]

Each of these regions may be made up of a set of components, according to one example embodiment, where each example component may correspond to a single example clinical lab value and/or the result of a functional test, etc., according to one example embodiment. (For details on example assignment of example lab values to regions, see the Appendix, according to one example embodiment.)

According to one example embodiment, an example reasoning behind this decision, to make clinical lab values and functional test results the fundamental unit of the MORRB and of its regions, may include the following: e.g., but not limited to, if the example MORRB, according to one example embodiment, is to be an accurate, detailed depiction of health, it should be based on the most advanced and precise information available, and for it to be meaningfully and easily interpreted by the user, this information should be localized and organized in an intuitive manner, according to one example embodiment. This line of reasoning, according to one example embodiment may take one to example design of the MORRB, according to one example embodiment. Naturally, the question of where example biometric information comes into play arises now, according to one example embodiment. Biometric information, according to one example embodiment, may be discussed in the next section (Dynamics), according to one example embodiment.

According to one example embodiment, example impact of certain environmental (e.g., toxins, regional geopolitics, etc.), psychological (e.g., mood, stress, etc.) and social factors (e.g., position in and stability of social network, etc.) to be diffuse and global, but not homogeneously distributed across the surface of the example MORRB, according to one example embodiment., According to one example embodiment, these factors may be translated, and the factor of advancing age, into the notion of elasticity, may be provided and/or displayed, in the context of the example MORRB, according to one example embodiment. In short, the more unhealthy these dimensions of the user's well-being are, according to one example embodiment, and the more the user ages, the more inelastic the surface of the MORRB may be depicted and/or may become in its illustrated example deformities, i.e. it may become easier to deform the example MORRB orb membrane, according to one example embodiment, but it may be found to be harder to reverse surface depressions, according to one example embodiment. This property of MORRB, according to one example embodiment, can be thought of as related to the concept of longevity escape velocity (LEV); it may become, according to one example embodiment, non-trivially harder, as time goes on or as environmental conditions worsen, to escape decline, according to one example embodiment. [NOTE: Determine how elasticity scalar field may be computed and periodically updated, according to one example embodiment.]

### Dynamics

As mentioned in the previous subsection (Structure), according to one example embodiment, the example structure of the example MORRB orb membrane, according to one example embodiment may be dictated by an example ensemble of received example clinical lab values and/or example functional exam results, according to one example embodiment. These data may give the example system, according to one example embodiment of the disclosed system, an example thorough picture of user health, according to one example embodiment. The issue is, in conventional common practice, according to one example embodiment, there may be a heavy overhead cost associated with taking lab tests and functional exams for the user, so these data can likely only be updated, at a maximum, a few times per year, according to one example embodiment. As such, the example MORRB, according to one example embodiment, may need a way to interpolate these values in the meantime, in order to update its state for historical reporting, in the absence of data at example requested depictions of data granularity, which may not be available, according to one example embodiment, and/or extrapolate for such example predictive features seeking to identify example potential what ifs, and/or trending of data over time for example predictive embodiments, according to one example embodiment. This is where the biometric data may come in, according to one example embodiment. Biometric data may be used to approximate clinical lab values and functional test results, in some embodiments, according to one example embodiment.

More formally, according to one example embodiment, the dynamics of the example MORRB can be described by two functions, which we can call f and g, according to one example embodiment.

“f” is responsible for approximating the clinical lab values and functional test results and it can be defined in the following way, according to one example embodiment:

f:L×F×B×C→(L′, F′) where L is the original clinical lab data, F is the original functional test results, B is the biometric data (both recent and historical), C is contextual data (e.g., medical history, diet, habits, etc.), L′ is the updated set of lab values, and F′ is the updated set of functional test results, according to one example embodiment.

“g” is responsible for taking these approximate lab values and test results and translating them into topology.

It can be defined in the following way:

g:L′×F′→M′ where M′ represents the updated MORRB topology, according to one example embodiment. (As an aside, f and g can be thought of as making up T, the state transition function introduced in the Overview of this section. More precisely, rolling f into g gives T, or T≡g(f)), according to one example embodiment.

[NOTE: need to determine specific algorithmic implementation of f i.e. how to approximate lab values/functional test results given biometric data and contextual information]

“f” can be implemented with an ensemble of machine-learning models, according to one example embodiment.

According to one example embodiment, one may determine specific algorithmic implementation of “g,” i.e., e.g., but not limited to, how to translate approximate lab values/functional test results into topology, according to an example embodiment.

According to one example embodiment, various example features may be included, such as, e.g., but not limited to:
introduce idea of inter-layer relationships, and inter-region relationships and outline the two major interactions (e.g., but not limited to, concept of stability, harsh grading problem, compensation problem, etc.), according to one example embodiment.
According to one example embodiment, one may focus on example inter-layer relationships and then later on, according to one example embodiment, one may potentially show how the ideas here can be extended to handle inter-region relationships. Various example embodiments may include, e.g., features to address, e.g., but not limited to,
   stability,
   harsh grading problem,
   compensation problem,
   global, inter-region relationships, etc.

### Example Software Embodiments

### Example Overview Embodiments

According to one example embodiment, various example features may be included, such as, e.g., but not limited to:
overview
database+wearable middleware+electronic medical record (EMR) middleware and/or APIs (within database, giving special attention to security, access rights and controls, etc.), according to one example embodiment;
various example different platforms including, e.g., but not limited to: web browser, client/server, peer-to-peer, touchscreen tablet, or virtual reality (VR)/augmented reality (AR)/mixed reality (MR) headset, and/or wearables interfaced, etc., UI/GUI/, according to one example embodiment, various example embodiments, may include using an example touchscreen and/or browser UI, and/or other example more immersive, example virtual reality rendering and/or AR/MR images inserted into an example user's field of view (FOV), etc., according to one example embodiment;
example apps (e.g., example mobile apps, e.g., various example OS support including Android, iPhone iOS, etc.); and within those, different wearables and e.g., but not limited to, electronic medical records, EMRs/electronic health records, EHRs/personal health records, personal health records PHR/and/or Epic EMR records, patient based health records, medical records, health records, and/or sensed data, etc., according to one example embodiment.

### Example Functionalities and/or Features Embodiments

According to one example embodiment, various example features may be included, such as, e.g., but not limited to:
according to one example embodiment, general functionalities of an example MORRB, according to one example embodiment; i.e. what users can do in various example embodiments may include:
model initialization, according to one example embodiment
add/remove data streams (users can personally balance privacy concerns with utility of model), according to one example embodiment
edit graphical settings, according to one example embodiment
observation, according to one example embodiment
view MORRB current state, according to one example embodiment
focus in example specific regions of MORRB/decompose regions+visualize intercomponent relationships/visualize global relationships, view histories, predict/extrapolate futures, according to one example embodiment
reflection, according to one example embodiment
create histories, according to one example embodiment
projection, according to one example embodiment
generate projections w or w/o hypothetical data (physically deform sphere to see non-local effects), according to one example embodiment
collaboration, according to one example embodiment
bring physicians/experts in, according to one example embodiment
share info with friends (e.g., musculoskeletal system, etc.), according to one example embodiment

### Example Architecture Embodiments

According to one example embodiment, various example features may be included, such as, e.g., but not limited to architectures and example data flows as set forth herein, according to one example embodiment.

### Example Electronic Medical Record (EMR) Adapter Embodiments

According to one example embodiment, various example features may be included, such as, e.g., but not limited to example electronic medical record devices and/or systems interfaced to example electronic databases, example sensors, and/or other example wearables such as smart watches, glucose monitors, blood sensors, etc., as set forth herein, according to one example embodiment.

### Example Wearables and Middleware Embodiments

According to one example embodiment, various example features may be included, such as, e.g., but not limited to, example wearables such as, e.g., smart watches, glucose monitors, blood pressure and/or pulse rate, and/or pulse rate oximeters, etc., monitors, blood sensors, EMR/HER/PHR systems and/or system interfaces, and/or application programming interfaces (APIs), etc., as set forth herein, according to one example embodiment.

### Example Database Embodiments

According to one example embodiment, various example features may be included, such as, e.g., but not limited to, example databases such as, DBMSs, flat file DBs, hierarchical DBS, relational DBs, SQL DBs, NoSQL DBs, graph databases, etc., as set forth herein, according to one example embodiment, and may include certain example services and/or compliant access controls such as, e.g., but not limited to, —special attention to security (HIPPA, GDPR), according to one example embodiment.

### Example Web Platform Embodiments

According to one example embodiment, various example features may be included, such as, e.g., but not limited to, example web and/or web-browser based applications, applets, JAVA applets, XML, HTML, etc., according to one example embodiment.

### Example Mobile Platform Embodiments

According to one example embodiment, various example features may be included, such as, e.g., but not limited to, example mobile application platforms including, e.g., Mobile OS, Mobile App, iOS, ANDROID, WINDOWS, PC OSs, UNIX OSs, TABLET OSs, open source applications, closed source applications, etc., according to one example embodiment.

### Example VR/AR/MR Platform Embodiments

According to one example embodiment, various example features may be included, such as, e.g., but not limited to, example mobile application platforms including, e.g., AR/VR/MR, OCULUS, VR HEADSET, APPLE VISIONPRO, MICROSOFT HOLOLENS, HYPERVR, MAGICLEAP, etc., according to one example embodiment.

### Example Infrastructure Embodiments

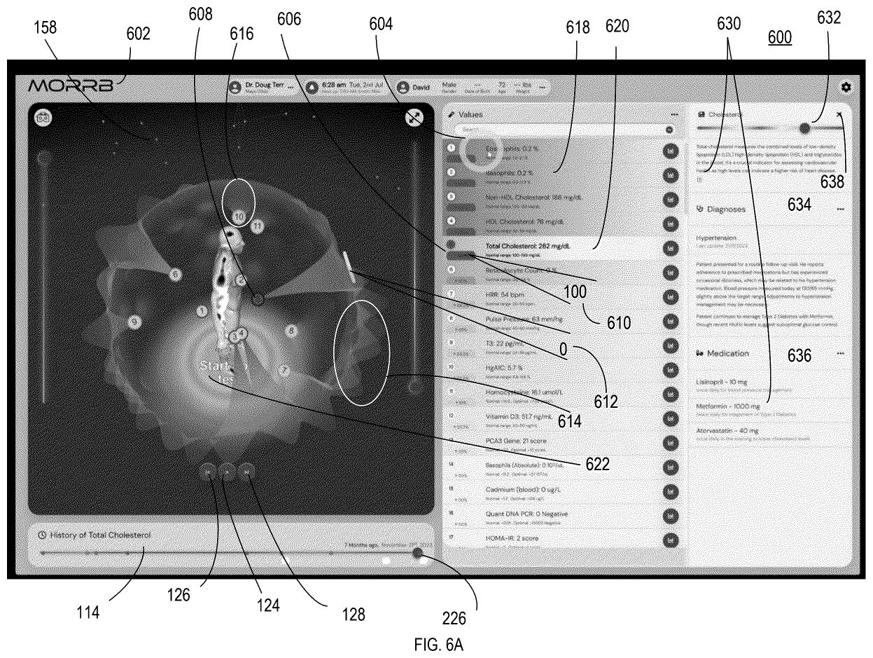

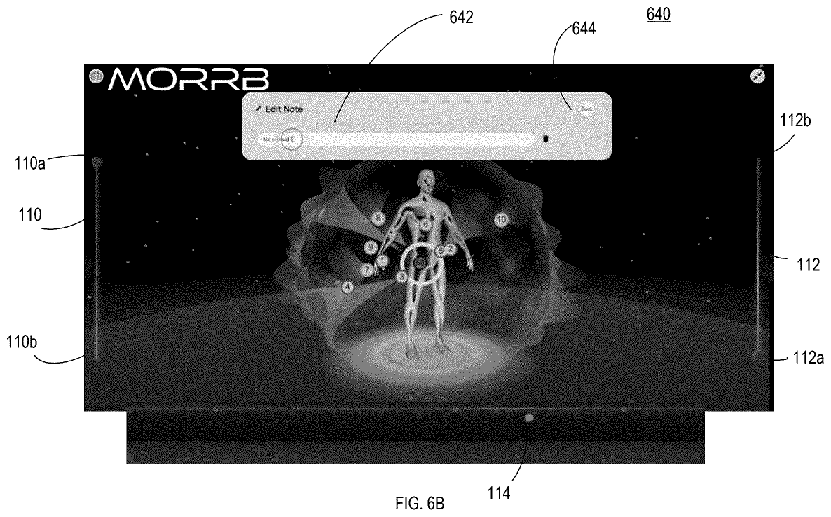

According to one example embodiment, various example features may be included, such as, e.g., but not limited to, a load balanced and/or web server, and/or application server as illustrated for example in FIG. 6, or other back end infrastructure may be used as an example service provider system, according to one example embodiment.

According to one example embodiment, various example features may be included, such as, e.g., but not limited to, —may include, e.g., but not limited to, example version control (and/or may include use of an example Github repository)—CI/CD, according to one example embodiment

### Alternative Example Embodiments

According to one example embodiment, various example features may be included, such as, e.g., but not limited to,
example complex patients, and/or other types of users, entities, patient users, etc., according to one example embodiment; and
example gamification, digiMe example digital me customizable and/or AI/ML intelligent avatar, and/or social media sharing features, and/or other viral sharing of all and/or part of a user's data, according to one example embodiment.

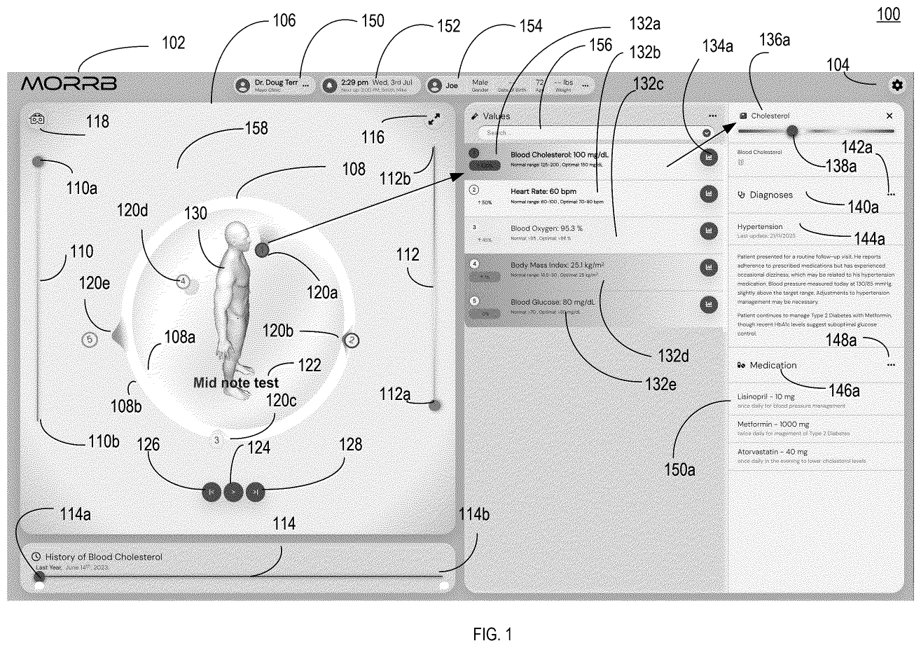

FIG. 1 depicts an example embodiment of an example screencapture 100 of an example spherical orb graphical user interface visualizer in an example light mode view of an illustrative example power user interface including an example uses avatar illustrated within, an example horizontal axis slider GUI element timeline, an example left vertical axis slider GUI element for varying time increments, and example right vertical axis slider GUI element for varying/filtering amount of severity data displayed in the visualizer, according to an example embodiment of the present invention.

FIG. 1 depicts an example “light mode” view 100 of an example screen capture of a graphical user interface (GUI) of an example Morrb computer software application program 102, according to one example embodiment. FIG. 1 may include as illustrated, an example graphical user interface (GUI) computer application software program 100, which may include, e.g., but not limited to, a title bar 102, and an interactive, GUI element, enabling interactive displaying (modifiable by use of a user mouse/pointing device, or touchscreen gesture) in an example two (2) dimensional rendering 106, of an example three (3) dimensional semitransparent, rotatable, spherical orb 108, representation as illustrated including example physical health test data of a given patient with a gender selectable avatar 130 (shown with an example male avatar) of the given patient which is depicted placed at least partially within, or adjacent, the transparent spherical orb 108 having an inner spherical surface 108a and an outer peripheral spherical surface 108b, actual observed data 120a, 120b, 120c, 120d, 120e displaying example externally concave indents, and convex peaks, and example inwardly concave peaks and convex valleys in the respective outer and inner surfaces of the orb 108, depending on the relationship between the actual observed data values, relative to and one or more normal ranges from example related minimal to maximal normal health metrics, assisting in graphically highlighting visually severity by, e.g., but not limited to, expanding the orb 108's external surface for beneficial values and decreasing the inner radius of the inner surface of the orb 108 for nonbeneficial values relative to minima of normal ranges and providing example color coding for levels of severity from green 132d, 132e for good or beneficial values, to neutral, 132b, 132c, to red 132a for nonbeneficial and severe or hazardous values for data approaching nonbeneficial levels of metrics based on relationship to normal ranges and indicates any of the physical health test data if it reaches hazardous levels, according to an example embodiment.

According to one example embodiment, notes 122 may be inserted, according to one example embodiment.

Various example axes scrollbar GUI elements 110 (from a default 110a to 110b), 112 (from a default 112a to 112b), and 114 (from 114a to 114b), may be provided including, e.g., but not limited to, data history scrollbar 114, which may enable user interactive scrolling through a history of a particular highlighted data element from earliest history 114a to latest actual captured history data 114b, according to an example embodiment. Change of data over time may be “played” by selection of play button 124, or rewinding to the beginning 126, or advancing to the end 128, according to one example embodiment.

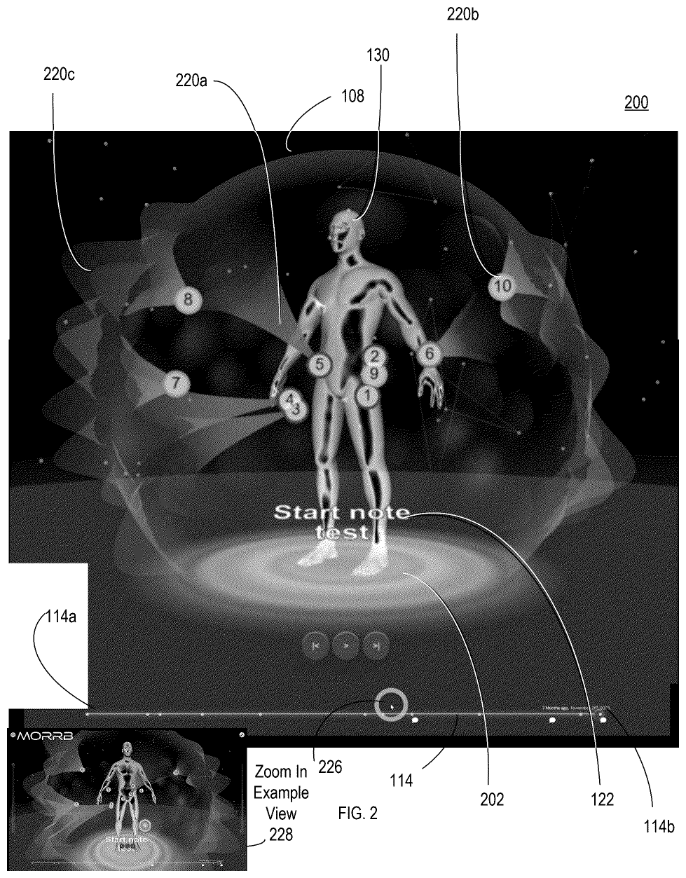

FIG. 2 depicts an example embodiment of an example screencapture 200 of an example spherical orb graphical user interface visualizer in an example dark mode view of an illustrative severity ranges view illustrating example membrane example deformations of example indicator intrusions and extrusions and forming an example volumetric envelope between the example membrane surface and including an example user avatar illustrated within, and example zoomed in example view of a zoomed in spherical orb visualizer UI, according to an example embodiment of the present invention.

FIG. 2 depicts an example “dark mode” view 200 of an example portion of the example Morrb application 100, which according to one example embodiment may include, e.g., but not limited to, a health visualizer program 100 that may provide an intuitive overview of a person's overall health, according to one example embodiment. Think of a car dashboard; with a quick glance, you have important indicators to help you operate the vehicle intuitively. A healthy person, represented by the avatar 130 may have a substantial cushion between the avatar 130 and the inner surface of the example spherical envelope orb 108, according to one example embodiment. For certain positive health data, the exterior surface 108b, 220c (which may be colored green) of the orb may expand as was shown in 120e, 123d, while for certain unhealthy data versus normal ranges, the inner surface 108a of the semi-transparent orb 108 may be illustrated to collapse inwardly an convex manner inwardly 220b (which may be colored yellow), or more severely as 220a (which may be colored red), 120a toward the avatar 130 representation of the person, according to one example embodiment.

Morrb application 100, according to one example embodiment may include a standalone software application program, which may allow a user to add information within the application and/or may allow importing data from saved Morrb data files, according to one example embodiment. The Morrb application 100 may also include, e.g., but not limited to, a developer-focused method of importing custom-structured comma-separated value files (CSVs) for common health indicators, according to one example embodiment.

The current example illustrated version of Morrb 100, according to one example embodiment, may include an example screen capture of a hypertext markup language (HTML) file that a user may access using an HTML browser and can be found by a user searching to find and selecting to open the file on the user's computer, according to an example embodiment. In one example embodiment, a majority of the program, according to one example embodiment, may be stored completely locally, aiding in its resiliency. According to other example embodiments, a mobile application, and/or a client/server based, and/or cloud-based, and/or software as a service (SaaS) cloud-hosted system, and/or Application Server hosted application and/or web server accessible system, which may alternatively be provided, according to other example embodiments, and/or may be programmed in another example programming language, according to other example embodiments.

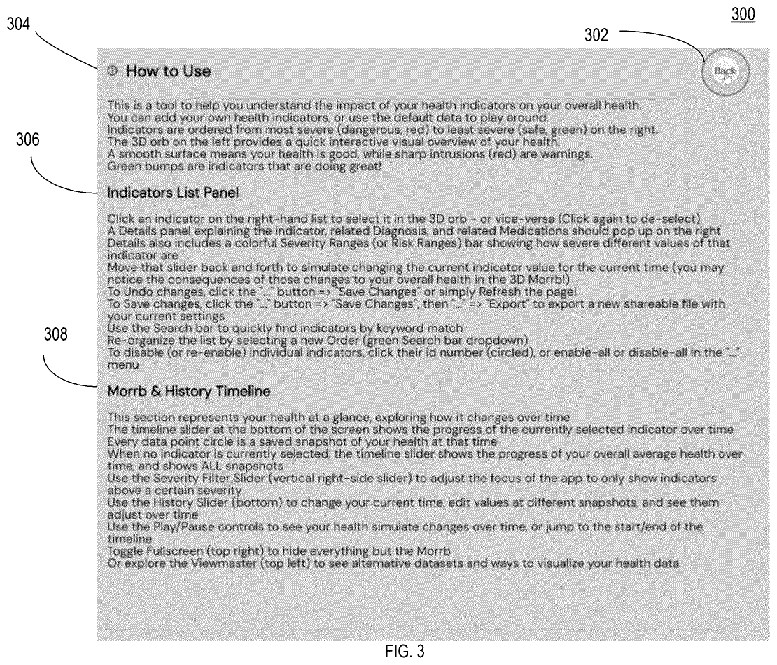

FIG. 3 depicts an example screen capture 300 of an illustrative example on-screen ‘How to Use’ tutorial displaying various help information, according to example embodiments of the present invention.

FIG. 3 depicts an example screen capture of an example on-screen ‘How to Use’ tutorial 300, which, according to one example embodiment, may display various information 304, 306, 308, via an example graphical user interface (GUI) example popup window, according to one example embodiment, upon the program 100 being loaded. When a user is satisfied, after having read the example displayed instructions, which may include, e.g., but not limited to, as illustrated, example how to use 304, indicators list panel 306, and morrb & history timeline 114 308 information, the user may choose to click the highlighted back button 302 on the example pop-up screen 300, and the application 100 may cause the popup screen to disappear, leaving the user at the GUI as shown in FIG. 1, according to one example embodiment.

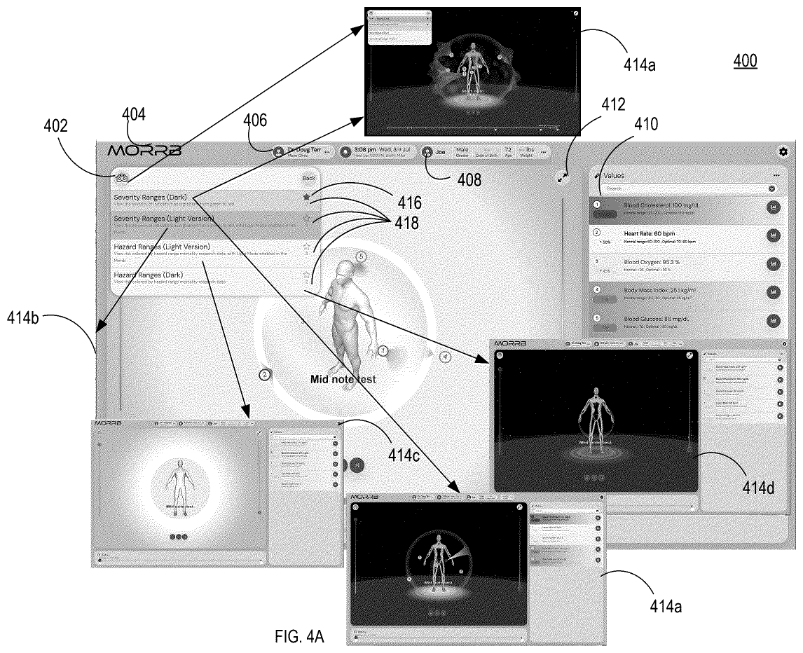

FIG. 4A depicts an example embodiment of an illustrative screencapture 400 of an example viewmaster selection menu including example favorite star elements and multiple numeric access counters and dark and light view, severity and hazard range view as well as example light mode severity views with example high, medium and low severity indicators for example sensed data graphically displayed on an example spherical orb graphical user interface, according one example embodiment of the present invention.

FIG. 4B depicts an example embodiment of an illustrative view 420 of an example viewmaster selection GUI button 118 of visualizer 414b including example female gender avatar 130a displayed on an example spherical orb membrane 108 graphical user interface 414b, with illustrative example timeline 414, example right vertical axis 112 from 112a to 112b illustrating color coded severity, resize button 116, application power user mode button 102, illustrative indicators 120a, 120c, and 120d, timeline 114 from 114a to 114b, play/pause button 124, rewind 126, and forward 128 buttons, according one example embodiment of the present invention.

FIG. 4A depicts an example diagram 400 in which, there are illustrated various example sections of the GUI display when we the example Morrb program 400 is loaded, according to an example embodiment. Each example area of the example user interface may be described in greater detail, below, after an initial overview of the screen 400, according to one example embodiment.

At the top, there are three areas in this view, according to one example embodiment. On the far left, there is a ViewMaster 402 for switching the display of information, according to one example embodiment. Next, we have the title 404 of the Morrb program, which, when clicked by the user, will display expanded views to show additional information for the physician 406, the patient user 408, e.g., at the top, and may show the indicator information and interactions 410 on the right, according to one example embodiment. At the top right, there is a button 412 that when a user selection is detected, may perform the same action as clicking on the Morrb name, i.e., displaying the information 406, 408, at the top, 410, etc., according to one example embodiment

The ViewMaster 402, when selected by the user, causes the GUI system to, e.g., display various example views of patient information 414a-d, according to one example embodiment. For example, a user may activate the menu by clicking on the ViewMaster button 402 at the top left, according to one example embodiment. Currently, according to one example embodiment, there are four example views 414a, 414b, 414c and 414d, and may be expanded to enable selection of many other example types of views, as the program adds additional example features, according to one example embodiment. A user can select a favorite view (or views) using favorite star GUI element 416, which may remain at the top, according to an example embodiment, for easy example user access, according to one example embodiment. There is an example counter 418 (see, e.g., but not limited to, numeric counter numbers “3” “3” “3” “2” indicating/displaying/providing for interactive access, an example numeric value of an example number of times that each view has been selected by the user, etc., in one embodiment) indicating, e.g., but not limited to, how many times a view has been selected, according to one example embodiment. The program, according to one example embodiment, may automatically sort views based on the decreasing number of selections of the counters 418 after the “starred” favorite views, according to one example embodiment.

We will cover the general concepts of the multiple views 414a-d, shortly; let's start with the basics for all Morrb views, according to one example embodiment.

As illustrated in FIGS. 1-4A and 4B, according to an example embodiment, front and center an example 3D model of an example person user patient may be illustrated representing an example patient 130, 154, according to one example embodiment. A person can choose their appropriate gender 130, 130a, which as illustrated shows an example male FIG. 130 (or a female gender as illustrated, for example in FIG. 4B avatar 130a, according to one example embodiment. According to other example embodiments (not shown) options may include, e.g., but not limited to, highlighting various parts and/or portion(s) of the person's illustrated model, user customization, choosing example illustrated user-selectable and/or customizable features, an example rigged model for animation, and/or the ability to zoom in for close-ups, etc., for additional example metrics on similarly located health groups, allowing for higher granularity of user selection and/or data information display, analysis, and/or access, according to various example embodiments.

Referring to FIG. 2, diagram 200, one may see displayed the Morrb surface 108, according to one example embodiment, called the Morrb Membrane 108, which, according to one example embodiment, may start as substantially a sphere and then an example inner and/or outer surface of the spherical orb may be deformed in 220a, 220b and out 220c based on example indicators (see e.g., but not limited to, numeric indicator labels 10 220b, 5 220a, 8, 7, 4, 3, 2, 9, 2, etc., and their severities, according to one example embodiment. Over time, using example timeline axis 114, 226, the user may view displayed system illustrated changes and may see the Morrb's indicator values and the surface change over time, as a user selection 226 of time varies, and indicating example severities (e.g., but not limited to, in color indication and/or other visible severity level indication, etc.) as any change, according to one example embodiment. The size of Morrb may change based on overall health, according to one example embodiment. A larger membrane may illustrate that there is more distance (a greater envelope of space surrounding the user 130 to the inner surface of the membrane 108) between the illustrated represented user person and the indicators 220a, 220b, that may come to cause the illustrated user harm, according to one example embodiment.

As illustrated in zoomed in example view 228, a user can select to use example GUI buttons to zoom in and out the example displayed image, and/or using the example mouse scroll wheel or pinch zoom on mobile, according to one example embodiment.

Also, as illustrated between FIGS. 1, 2 and 4, a user can select to rotate the example virtual camera display view by, according to one example embodiment, clicking and/or dragging the mouse left and right, as well as up and down, according to one example embodiment.

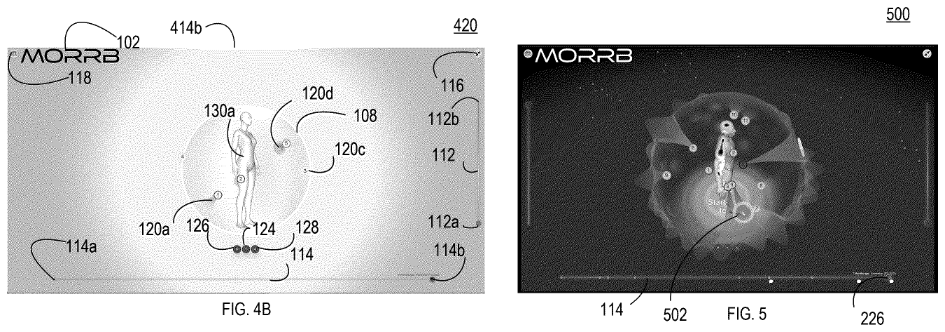

FIG. 5 depicts an example embodiment of an example dark mode view 500 of an example illustration of an example spherical orb membrane user interface with example indicator intrusions and extrusions illustrating using example color-based severity example indications, and defining an amorphous three dimensional envelope volume between the example inner surface of the membrane and the illustrated example user avatar and illustrating by proximity example health data with detrimental sensed data appearing proximate in distance with example intrusions coming near to the avatar, and including a timeline GUI element for browsing backward or forward through various timestamped sensed data, according to one example embodiment of the present invention.

FIG. 5 depicts an illustration 500 further showing, according to one example embodiment, a user may click on the example membrane, according to one example embodiment, may select and/or deselect the closest indicators 4 as shown, according to one example embodiment. The example virtual camera may also rotate the virtual camera's point of view (POV) to provide an example better view of the selected indicator, according to one example embodiment.

FIG. 6A depicts another example embodiment of an illustrative screencapture 600 of an example dark mode view of an example spherical orb membrane user interface with example indicator intrusions and extrusions illustrating using example color-based severity example indications, additional severity level variation illustrations, example outer membrane extrusion positive indicators, and defining the amorphous three dimensional envelope volume between the example inner surface of the membrane and the illustrated example user avatar and illustrating by proximity example health data with detrimental sensed data appearing proximate in distance with example membrane intrusion indicators coming near to the avatar, and including a timeline GUI element for browsing backward or forward through various timestamped sensed data, example play/pause button, example jump to first data, example jump to last data, example indicator scroll bars, additional details, diagnoses section, medication section, visualizer and indicator list including severity color-based indicators and numeric severity metric values, and example note area, example starry background in example view, according to one example embodiment of the present invention.

FIG. 6A depicts an example illustration 600, where for a given indicator 608, 604, after selection of the title 602 button, each indicator 604, 608, 606, according to one example embodiment, if shown, may have an example metric called ‘severity,’, according to one example embodiment. An example severity level, according to one example embodiment, may range from, e.g., but not limited to, 0 (612) to 100 (610, 608), according to one example embodiment. 0 may be the lowest (at the level of the spherical membrane orb 612) and may be most optimal, according to one example embodiment. An example severity level of 100 610, 606, 608 may indicate the patient user may be on an example cliff's edge of a potentially near terminal situation marked by the associated indicator 608, 606 with severity 610, according to one example embodiment.

Referring back to FIGS. 2 and 6A one may see illustrated example indicators, now to be explained with a little more detail, according to one example embodiment. On the example displayed membrane 108 there can be example intrusions and extrusions of the membrane (e.g., but not limited to, example concavities, conical hole portions, and/or indents, or convexities, example outward conical portions, and/or protrusions extruding from the example surface of the example membrane of the example spherical orb, etc.), according to one example embodiment. Extrusions that expand out of the membrane 614 may be color coded, such as, e.g., but not limited to, may be displayed in an example green (e.g., for good indication, etc.) which may indicate an example low severity level 612, 614 example indicator for a particular health indicator 614, according to one example embodiment. Along the surface of the example membrane 108 the example extrusions may turn more to another example color such as, e.g., but not limited to, an example transparent yellow hue 616 (In Dark mode), etc., according to one example embodiment. The example intrusions, according to one example embodiment, may be color-coded with another color such as, e.g., but not limited to, may turn an example red color 608, (and/or may have another type of example highlighted appearance, e.g., for review and recognition by a colorblind individual, such as, e.g., but not limited to, blinking, highlighting, dotted lines, etc.), according to other example embodiments. The closer the example intrusions 608 may be in proximity to the illustration of the example person patient user, the higher the example indicator's example severity level 610, 606, according to one example embodiment. The sensor detected data may be displayed in intuitive graphical fashion, to assist the patient in graphically visualizing the severity of certain example conditions, according to one example embodiment. For example, according to one example embodiment, if the example intrusion indicator is displayed substantially near and/or touching and/or proximate the example person user patient illustration, according to one example embodiment, then that may indicate that the person user may be according to one embodiment, on an example edge of death, see e.g., indicator 5 608, based on the electronic analysis and electronic comparisons of sensed indicator values as compared to expected normal ranges of values, and may illustrate visually in the example display, e.g., the severity level and/or the importance, and/or the relevancy of, the example biomarker indicator to the patient person user's health, according to one example embodiment. According to an example embodiment, the patient person user's goal may then be to take steps in the user's life and activities to assist in e.g., ensuring that none of these example biomarker indicators 608, 604 ever will be displayed to get near enough in their display in proximity to the illustrated user, so as to e.g., touch or be displayed relatively near the illustrated digitized person of the patient user, according to one example embodiment. Physicians, according to one example embodiment, may work with patients (e.g., on medical interventions, and/or therapies, and/or suggested changes in behavior and/or patient activity, etc.) and come up with example suggested plans, therapies, treatments, and/or interventions, etc., on how to push back those intrusions toward a more health illustrated normal range of indication, according to one example embodiment.

Each example indicator 608, being displayed, according to one example embodiment, may have a number (e.g., an electronically stored numerical value,) associated with the example indicator, made visible on the example display when the example indicator list is opened by user selection of 602, according to one example embodiment These numbers/numeric values may have a border 618 that may share the same example color indication (e.g., green, red 618, yellow) as the indicator 608 severity level color code 610, 606, according to one example embodiment. When an indicator 606 is selected, the example border 620, according to one example embodiment, may turn, e.g. but not limited to, gray, etc. and the example background color may become displayed as, e.g., but not limited to, the example severity color, according to one example embodiment. This example display illustration technique of using example color coding may also be true on an example mouse hover and/or other user gesture, when the user is about to click on an example indicator using the example user interface, when the example user's pointer, such as mouse arrow and/or touchscreen-sensed pointer or cursor location, according to one example embodiment.

According to one example embodiment, example embodiments may also include, e.g., but not limited to, example information 622 that may be example displayed generalized notes (e.g., see Start Note Test 622, etc.), etc., through the patient timeline, according to one example embodiment. For example, an example note may signify, according to one example embodiment, that the example person user's example Morrb spherical orb GUI element may be skewed from the last batch of data due to, e.g., but not limited to, a recent foodborne or other illness, etc., according to one example embodiment. The example display may be provided showing up visually in its display proximate to, or near the person user's illustrated feet 622 and/or at another example location, and may include, e.g., but not limited to, example useful notes added to the example patient information, according to one example embodiment. These example textual note(s) information 622 may help provide context for the physician and patient user, according to one example embodiment.

As illustrated in FIG. 4A, the example program 400, according to one example embodiment, may offer both an example Light Mode 414b, 414c and an example Dark Mode 414a, 414d, according to one example embodiment, and each of those light and dark modes has a Severity Ranges mode 414b, 414a, respectively and a Hazard Ranges mode 414c, 414d, respectively, according to one example embodiment.

Example Light Mode 414b, 414c shows an example streamlined UI with shallow green and red deformations of the Morrb Membrane 108, the light mode is subtle and may merge well with the rest of the white-themed UI as illustrated in FIG. 1, according to one example embodiment.

Example Dark mode 414a, 414d shows an example illustrated person avatar displayed as shown in an example starry void of FIG. 2, according to one example embodiment. There may also be displayed, as shown, an example floor 202, that may include example color coding, which may serve as an example at-a-glance indicator of an example overall health, which may include, e.g., but not limited to, color, animations, audio and/or visual content, etc., according to one example embodiment.

See FIG. 5, screencapture 500 illustrating when, e.g., but not limited to, a mouse and/or pointer, etc., hovers over the example membrane 108, the intersection 502 between the mouse and membrane 108 may be displayed as illustrated, as shown for easy tracking of mouse position., according to one example embodiment.

Additionally, the closest indicator (e.g., 3, 4, or 7, etc.) for interaction may turn white or another example color and/or indication (this may change with alternative embodiment animations, according to other embodiments), according to one example embodiment.

According to one example embodiment, an animated line may illustrate (not shown) or indicate where a user's mouse position is and may flow to the item the use selects if the user were to click the mouse pointer and/or button, according to one example embodiment.

FIG. 6B depicts another example embodiment of an illustrative screencapture 640 of an example dark mode view of an example spherical orb membrane user interface illustrating the example horizontal and vertical sliders of the example visualizer and example note adding popup data entry window, according to one example embodiment of the present invention.

### Example Embodiment of an Example Timeline 114

At the bottom of the example application 100, there may be displayed an example timeline 114, 226 (see FIGS. 1, 2, 5, and 6A and 6B) where the user can see an example aggregated view where all of the patient user's data is shown, each example dot represented an example sample of data, arranged chronologically, according to one example embodiment. When no indicator is selected, according to one example embodiment, the example user interface may display all information for all indicators, according to one example embodiment. When an indicator is selected the timeline 114 may, according to one example embodiment, only show information based on that selected indicator, according to one example embodiment. A user may select to move to different points in time by selecting the slider GUI element using the user's mouse pointer and/or by long pressing and sliding on an example touchscreen display, according to one example embodiment.

As illustrated in FIG. 5, on the timeline 114, 226, one may see displayed miniature representations of the quantities and severities of data for a patient's health history, according to one example embodiment. A user, according to one example embodiment, can click or slide anywhere on the slider to move to another the displayable shown time for all indicators and metrics on the indicators list as well as the Morrb itself, according to one example embodiment. The indicators and metrics may always match, according to one example embodiment. While the user interface is fixed at an example specific time, such as, e.g., but not limited to, a time shown in FIG. 5, according to one example embodiment, the user such as, e.g., but not limited to, the patient user, and/or physician and/or authorized user can, e.g., but not limited to, modify information 618, 620, 630, such as, e.g., but not limited to, add and/or modify, revise, etc., data points and/or included notes, according to one example embodiment.

As shown in FIG. 5, according to one example embodiment, an example playback feature may be included so users can watch how an example tracked person's data indicative of the patient's health evolves over time, according to one example embodiment. This modifies the currently viewed time. A play/pause button 124 as well as ‘go to start’ 126 and ‘go to end’ 128 buttons and indicators 632 may reflect in their display their stored valued, for the example current time, according to one example embodiment.

FIG. 6B illustrates a screencapture 640 of the example GUI of the example MORRB application, in one example embodiment, how a user can also enter notes in response to an example timestamped edit note prompt, placed on the timeline 114, 226 for easy adding of information that will show up on the Morrb in the text box mentioned earlier, which may appear at the illustrated patient avatar's example feet position, according to an example embodiment.

Continuing with diagram 640 of FIG. 6, the left side may display an example vertical slider GUI element 110 that may indicate the example resolution of the time slider, which may be varied by user interaction with the GUI slider 644 selection, according to an example embodiment. The more the slider 110 is moved down toward example position 110b, the more is displayed indicator information nearer in time, according to an example embodiment. This may allow for much greater resolution when a user wishes to interact with and view displayed data that is really proximately close to other data in time, at a given level of granularity, without as much crowding of the data points on the timeline 114, according to an example embodiment.

Continuing with diagram 640 of FIG. 6B, as illustrated as displayed on the example screenshot 640, on the right side there may be displayed a user-interactive vertical slider GUI element 112, according to an example embodiment, that displays which of the indicators will be shown by filtering out various levels of severities. Default 112a (the bottom position), according to an example embodiment, may show all of the indicators, according to an example embodiment. By sliding slider 112 up toward 112b, slider 112 may filter out the lowest severities to make the displayed appearance of the various shown data of the Morrb user interface, appear less busy, i.e., with less information being shown, according to an example embodiment. The vertical slider 112 may add to the case of user interaction with the GUI and viewing of the display of only the most severe indicators, according to an example embodiment.

### Example Embodiment of an Example Power User Interface

In order to access the example power user interface, a user may select or use the user's mouse to click on the MORRB title bar 102, and by select the Morrb text button 102 at the top left, the power user interface, as illustrated in FIG. 1, described above, may be displayed, according to an example embodiment.

Upon loading the power user interface, the interface may display additional options of information no displayed in FIG. 6B, for example, including, e.g., but not limited to, physician information (Dr. Doug Terrence, Mayo Clinic, etc.) 150, etc. At the top of screenshot 100 of the example power user interface shown in FIG. 1, we see displayed, e.g., the physician information of the doctor currently interacting with and viewing the example Patient data displayed of patient 154 for example, according to one example embodiment. The example physician button may be coupled and/or connected to example additional features such as, e.g., but not limited to, access to that physician's patient health records, electronic medical records, billing systems, communications tools, electronic communication interfaces such as, e.g., but not limited to, electronic mail (email), short message system (SMS), multimedia message system (MMS), videoconferencing, telephone audio and/or video services, etc., according to various example embodiments. As is also displayed in screencapture 100, one may also see displayed on the example GUI, an example current time and/or date and/or time (such as, e.g., 2:29 pm, Wed, 3rd of July in FIG. 1, or 6:28 am Tues, 2nd July in FIG. 6A, etc.) 152, and the example next patient name and time as illustrated (e.g., “Next up 7:00 AM, Smith, Mike, etc.), and the current patient information 154, etc. We also see a snapshot of the patient information 154 such as Name (e.g., David, Joc, etc., image, (if available or a symbol or avatar, etc.) Gender (e.g., Male 130, Female 130a, etc.), Date of birth (if known or available may be shown), age (if known or available), and weight (if known or available), etc.

There is further illustrated in FIG. 1 screen capture 100, an example opened list of indicators 132 including various numbered indicators 132a, 132b, 132c, 132d, and 132c, etc., according to an example embodiment.

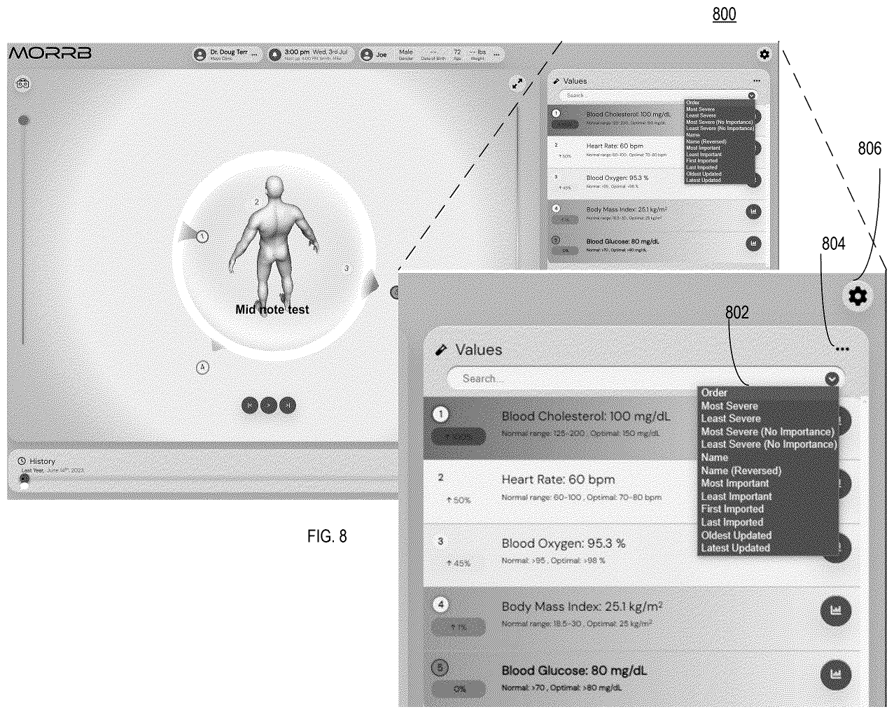

This indicator menu query field 156 may allow example searching and/or sorting of example indicators using the drop-down menu for sort options, see for example FIG. 8.

Each indicator 132 may have a number associated with the indicator for easy tracking within Morrb user interface 100, according to an example embodiment. Each example indicator number 1-5 will correspond to the numbers 1-5 120a, 120b, 120c, 120d, and 120e in the visualizer section 158, according to an example embodiment. The example severity number for a respective indicator is located directly below the number identifier of the respective indicator, according to an example embodiment, and as discussed with reference to FIG. 6A above, recall the example severity numbers may range from 0% 612 to 100% 610, in all views, according to an example embodiment. Reviewing screenshot 600 of FIG. 6A, one may see illustrated as displayed in the example GUI screen, the example name of the indicator 618, 620 and the current value and units, if available and/or for which data has been captured, for, e.g., the current time, according to an example embodiment. We also may see, e.g., but not limited to, the example Normal ranges for the given example indicator and/or biomarker and/o metric, and example Optimal ranges, if available for that example indicator in this view, according to an example embodiment.

By clicking on the indicator 620 such as indicator 620 of screen 600, or an indicator 608 from the visualizer 158, a new menu 632 may open up, according to an example embodiment. This may display as shown in 600, an example slider 632 that may allow the example user, to update the values for, e.g., but not limited to, the current timestamp for the selected indicator, according to an example embodiment. Click on the same indicator 620 on the UI 620 or the visualizer 158, or the ‘X’ 638 on the expanded indicator information 630, to close this indicator 630, according to an example embodiment.

In this same menu, a temporary listing may be displayed, as shown, for Diagnoses 634 and Medications 636 that maybe be associated with the selected indicator, according to an example embodiment.

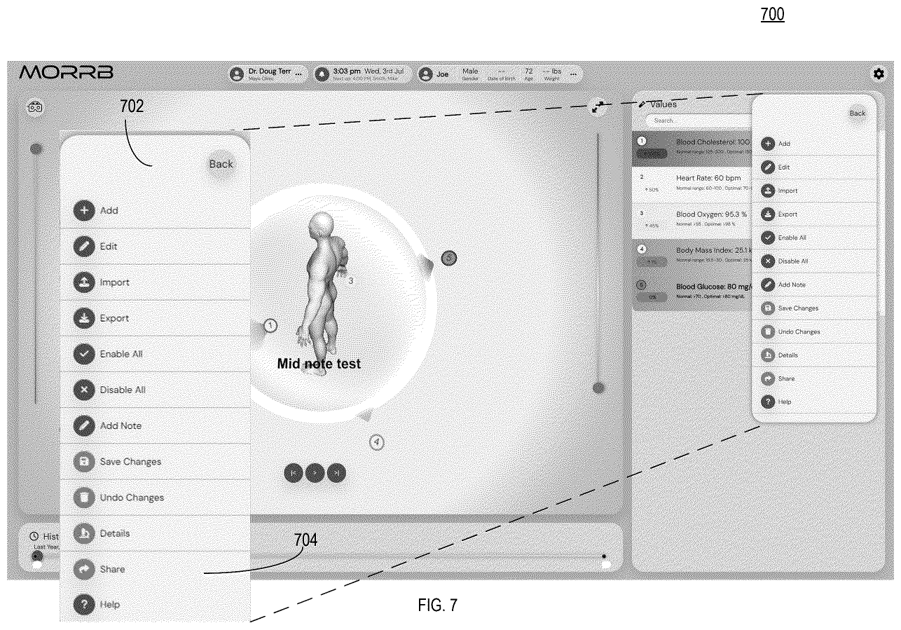

FIG. 7 depicts an example embodiment of an illustrative screencapture 700 of an example indicator addition/editing/enabling/disabling menu, according to one example embodiment of the present invention.

After selecting the ‘X’ 638 in FIG. 6B, the top right of the indicator menu may display on its “values” a three dot menu object ( . . . ) 804 (shown in FIG. 8, where the example three dots GUI element may be used to bring up an example menu 702 as shown in FIG. 7, or by using the pull down arrow of the search field, the menu of FIG. 8 may be displayed, according to an example embodiment. The menu 702, 802 choices displayed in 700 of FIG. 7, 800 of FIG. 8, respectively, allow the user to interact to select any of various example different listed optional menu features 702, 700 including, e.g., but not limited to, add, edit, import, export, enable all, disable all, add note, save changes, undo changes, details, share, help, etc. for FIG. 7, or drop down list of menu features 802, 800 for sorting, FIG. 8 may list example menu options 802 including, e.g., but not limited to, order, most severe, least severe, Most Sever (No Importance), Least Severe (No Importance), Name, Name (Reversed), Most Important, Least Important, First Imported, Last Imported, Oldest Updated, Latest Updated, etc., according to an example embodiment.

From these example menus 702 as displayed in FIG. 7, a user can, e.g., but not limited to, add, an example indicator, edit the current indicator, import, export, enable all indicators, disable all indicators, add a note, save changes, undo changes, open ‘How to Use,’ (help), and other example options for details and share, etc., all among other features, according to an example embodiment.

FIG. 8 depicts an example embodiment of an illustrative screencapture 800 of an indicator sorting options pulldown menu, according to one example embodiment of the present invention.

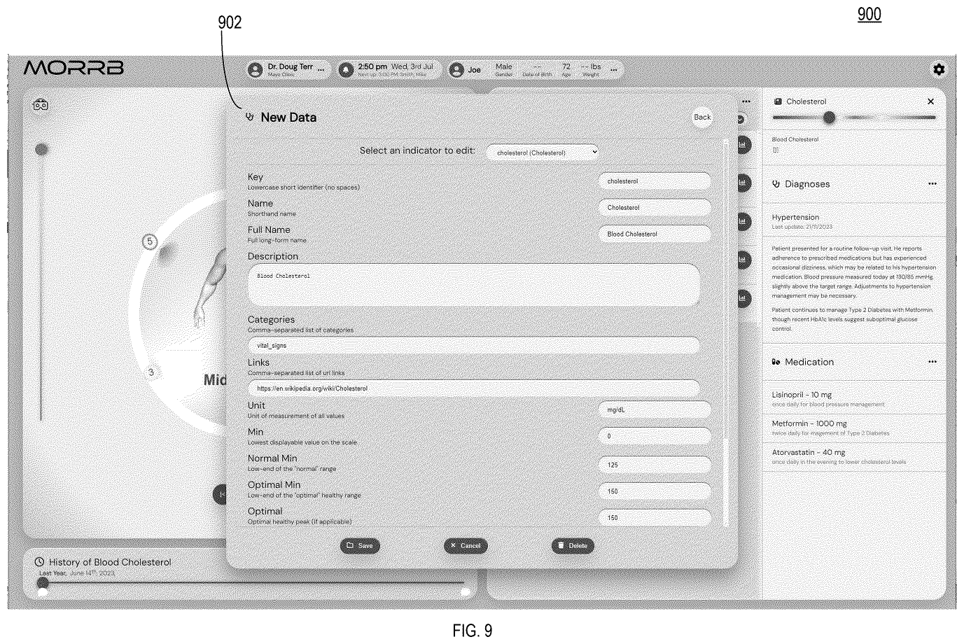

FIG. 9 depicts an example embodiment of an example screen capture 900 of an example edit a given example cholesterol indicator with example indicator biomarker details example fields popup window, according to an example embodiment of the present invention.

According to another example embodiment, an example GUI may include, e.g., but not limited to, an example chat element that allows various example electronic physician and artificial intelligence (AI)-based example interactions, including, e.g., but not limited to, e.g., interacting via, e.g., natural language search, natural language processing, voice recognition, speech recognition, speech generation, an interactive generative pre-trained machine learning (ML), deep learning based large language model (LLM) enabled, deep learning, convolutional neural network (CNN)-enabled, neural network, rules-based, heuristics, statistics based analysis, expert system, decision support system, AI/ML, based knowledge-based interactive library of constantly growing indicator information, and chatbot, user interaction based features, according to various example embodiments.

In other example embodiments, the example system may provide, e.g., but not limited to, functions for enabling creating example custom (and/or predefined) groupings that may allow for quick selection of example indicators and/or groups of indicators, and/or packages of prepackaged biomarkers and/or sensor-based related features such as, e.g., but not limited to, a wearable based sensor, an internet of things (iOT) based device, and/or intelligent sensors-based data and related analysis, data capture, data analysis, storage, etc., according to an example embodiment. An example is the example circulatory system indicators as a quick group selection, according to an example embodiment. FIG. 9 depicts screencapture 900 of an example capture of new data for an example Cholesterol indicator including an example new data menu 902 which may include, e.g., but not limited to, a way to capture and setup, and/or modify an example cholesterol indicator, including, e.g., but not limited to, example ranges from example minimum to example maximum, example units, link information, category information, optimal, optimal minimum, optimal maximum, normal minimum, normal maximum, importance, percentage severity value, etc., according to an example embodiment.

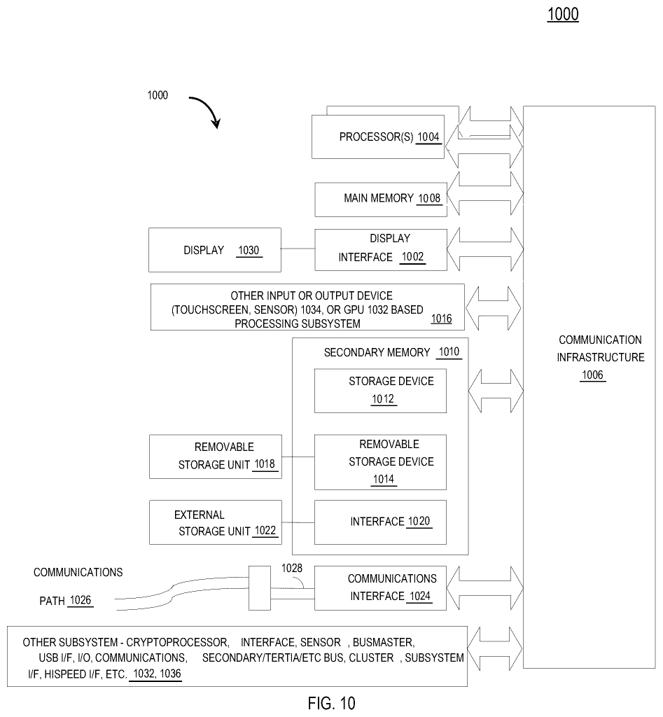

FIG. 10 depicts an example embodiment of an example schematic diagram 1000 of an example illustration of an example computer hardware architecture block diagram of an example component of any of various example devices of an example system, according to one example embodiment of the present invention.

FIG. 10 depicts an example embodiment of an example block diagram 1000, discussed further below with reference to the figure in greater detail, directed to an example computer system platform as may underly any of various example computing and communications devices according to exemplary devices of an example system, according to an example embodiment of the present invention.

FIG. 10 depicts a schematic illustration of an example communications and/or computing system 1000 implemented according to an exemplary embodiment. The system 1000 can include at least one processing element 1010, for example, a central processing unit (CPU). According to an exemplary embodiment, the CPU is coupled via a bus 1006 to a memory 1020. The memory 1020 includes, in an exemplary embodiment, a memory portion 1022 that can contain instructions that when executed by the processing element 1010 can perform the methods described in more detail herein. The memory 1020 may be further used, according to an exemplary embodiment, as a temporary storage element for the processing element 1010, and/or other uses, as the case may be. The memory may comprise, in an exemplary embodiment, volatile memory such as, e.g., but not limited to, random access memory (RAM), and/or non-volatile memory (NVM), such as, e.g., but not limited to, Flash memory, etc. Memory 1020 may further include, in an exemplary embodiment, a memory portion 1024 containing an application program and/or application data, etc., according to an exemplary embodiment. The processing element 1010 may be coupled to an input 1016, in one exemplary embodiment. The processing element 1010 may be further coupled with a database 1030 and/or other storage device 1030. Database system and/or storage device 1030, in an example embodiment, can be used for the purpose of holding a copy of the method executed in accordance with the disclosed technique. Database 1030 may further include, e.g., but may not be limited to, a storage portion 1034, which may include and/or contain sub-portions of an application, and/or data referenced by the application, in an exemplary embodiment. In one embodiment, the promotion system can be configured to execute the methods described herein with respect of the remaining figures. The exemplary method, system, and/or computer program products, may be hardwired or, presented as a series of programmable instructions to be executed by the processing element 1004.

The principles disclosed herein can be implemented as hardware, firmware, software or any combination thereof. Moreover, the software can be implemented as an application program tangibly embodied on a program storage unit or computer readable medium. The application program may be uploaded to, and/or be executed by, a machine comprising any suitable architecture. The machine may be implemented on a computer platform 1000 having hardware such as, e.g., but not limited to, a processing unit (“CPU”) 1004, a memory 1008, and/or input interfaces 1002, 1016, 1020, 1024, output interfaces (not shown), as well as other components not shown for simplicity, but as would be well known to those skilled in the relevant art. The computer platform may also include, in an exemplary embodiment, an operating system and/or microinstruction code. The various processes and/or functions described herein may be either part of the microinstruction code and/or part of the application program, and/or any combination thereof, which may be executed by a CPU 1010, whether or not such computer and/or processor is explicitly shown. In addition, various other peripheral units may be connected, and/or coupled, to the computer platform such as, e.g., but not limited to, an additional memory unit 1026 and/or removable memory unit 1026, an additional data storage unit 1036 and/or removable storage unit 1036, and a printing unit, and/or display unit, and/or other input 10100, output 1060, communication 1070 and/or networking components 1070, etc.

References to “one embodiment,” “an embodiment,” “example embodiment,” “various embodiments,” “exemplary embodiment,” “exemplary embodiments,” etc., may indicate that the embodiment(s) so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct or indirect physical or electrical contact with each other. “Coupled” may mean that two or more elements are in direct physical or electrical contact. However, “coupled” may also mean that two or more elements are not in direct contact with each other, but yet still co-operate or interact with each other.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these nontransitory signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” can refer to any device or portion of a device that processes electronic data from registers and/or memory to transform that electronic data into other electronic data that can be stored in registers and/or memory. A “computing platform” can comprise one or more processors. In one embodiment, a processor can include an embedded processor, and/or another subsystem processor, and/or a system on a chip, device.

Embodiments may include apparatuses for performing the operations herein. An apparatus may be specially constructed for the desired purposes, or it may comprise a general purpose and/or special purpose device selectively activated or reconfigured by a program stored in the device.

Computer programs (also called computer control logic), may include computer application programs, and can include object-oriented computer programs, and can be stored in memory 1020, and/or secondary memory, such as, e.g., storage 1020, 1022, 1024, 1026, 1030, 1034, 1036 and/or removable memory and/or storage units 1026, 1036, also called computer program products. Such computer programs, when executed, may enable the computer system 1000 to perform the features as discussed herein. In particular, the computer programs, when executed, may enable the processor 1010 to provide various functionality to the system 1000 so as perform certain functions, according to an exemplary embodiment. Accordingly, such computer programs may represent controllers of the computer system 1000.

In another exemplary embodiment, the methods may be directed to a computer program product comprising a computer readable medium having control logic (computer software) stored therein. The control logic, when executed by the processor 1010, may cause the processor 1010 to perform features as described herein. In another exemplary embodiment which can be implemented using software, the software can be stored in a computer program product 1036, 1026, and can be loaded into computer system 1000 using, e.g., but not limited to, the storage 1030, the removable memory and/or storage device 1026, 1036, respectively, hard drive and/or communications and/or network interface 1070, and/or router, etc. The control logic (software), when executed by the processor 1010, can cause the processor 1010 to perform the functions as described herein. The computer software can run as a standalone software application program running atop an operating system (OS), or may be integrated into the operating system and/or application program, and/or may be executed as an applet, or networked and/or client-server, and/or browser-based and/or other process as is well known.

In yet another embodiment, implementation may be primarily in hardware using, for example, but not limited to, hardware components such as application specific integrated circuits (ASICs), or one or more state machines, etc. Implementation of the hardware state machine so as to perform the functions described herein will be apparent to persons skilled in the relevant art(s).

In another exemplary embodiment, as noted, implementation can be primarily in firmware.

In yet another exemplary embodiment, implementation can combine any of, e.g., but not limited to, hardware, firmware, and software, etc.

Exemplary embodiments may also be implemented as instructions stored on a machine-readable medium, which may be read and executed by a computing platform to perform the methods described herein. A machine-readable medium may include any mechanism for storing or transmitting information in a form readable by a machine (e.g., a computer). For example, a machine-readable medium can include read only memory (ROM); random access memory (RAM); magnetic disk storage media; optical storage media; flash memory devices; electrical, optical, acoustical or other form of nontransitory propagated signals (e.g., carrier waves, infrared signals, digital signals, etc.), memory 1020, storage 1030, and others.

The exemplary embodiments make reference to wired, and/or wireless networks. Wired networks can include any of a wide variety of well known means for coupling voice and data communications devices together. Similarly, any of various exemplary wireless network technologies may be used to implement the embodiments discussed. Specific details of wireless and/or wired communications networks are well known and are not included, as will be apparent to those of ordinary skill in the relevant art.

All examples and conditional language recited herein are intended for pedagogical purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, communications and/or computing device, and/or computer, etc. It is further contemplated that the present invention may be run on a stand-alone computer system, and/or may be run from a server computer system that can be accessed by a plurality of client computer systems interconnected over a network such as, e.g., but not limited to, an intranet network, internet network, etc., and/or that is accessible to clients over the global Internet, etc. In addition, many exemplary embodiments of the present invention may have application to a wide range of industries, according to an exemplary embodiment. To the extent the present application discloses a system, the method implemented on a system, as well as a computer program product, such as, e.g., but not limited to, software instructions stored on a computer-readable/accessible nontransitory storage medium and executed on an electronic computer processor as a computer program to perform various steps of the method on a special purpose computer and/or in communication with other communication network devices including distributed mobile devices over one or more communication networks which may include wireless communication networks, etc., are within the scope of the present invention, according to an exemplary embodiment. Further, to the extent the present application discloses a method, a system of apparatuses configured to implement the method are within the scope of the present invention, according to an exemplary embodiment.

FIG. 10 depicts an example computer system hardware architecture diagram of a computer which may be used as a component of elements of the example systems of various described system and apparatus embodiments.

FIG. 10 depicts an exemplary schematic block diagram 1000 illustrating an exemplary computer/communications device hardware architecture as may be used in various exemplary components of an exemplary embodiment including user devices, patient devices, physician devices, diagnostic devices, cloudbased devices, client devices, server devices, network devices, routers, tablets, smartphones, authentication devices, hub, gateway, or switch devices, example computing devices of users, mobile devices, network communication components, routers, gateways, etc., computer device(s), payment service provider devices, third party or interfacing other computing or communications devices, routers, application servers, web servers, cloud-based computing devices, communication devices, switches, routers, gateways, telephone devices, mobile devices, content servers, web servers, database servers and/or other application servers, enduser devices, operations center devices, client and/or server devices, network routers, gateways, access points, and the like, according to various exemplary embodiments.

FIG. 10 depicts an exemplary schematic block diagram 1000 illustrating an exemplary computer/communications device hardware architecture as may be used in various exemplary components of an exemplary embodiment including mobile devices, wearables, cloud-based computing devices, content servers, web servers, database servers and/or other application servers, according to various exemplary embodiments. Diagram 1000 may include any of various exemplary computer systems as may be used as an external computer device, an internal and/or set top box (STB) or system on a chip (SOC), a mobile device, a wearable, a client, or server, web server, application server, and/or any other of the computing devices included in the other drawings, according to various exemplary embodiments. FIG. 10 depicts an exemplary diagram 1000 illustrating an exemplary computer/communications device hardware architecture as may be used in various components of exemplary embodiments of the present invention. FIG. 10 depicts an exemplary view 1000 of exemplary computer systems such as those represented logically in FIGS. 1-9, 11, 12, etc., including devices which may execute various modules, including components, systems, devices, computers, processors, mobile devices, routers, gateways, network devices, other computing and/or network devices, network monitoring and security operations data analysis computing devices which may execute various operating system and software components on such computing devices and/or subsystem devices and hardware/software modules as may be represented by devices implied by, or perform steps illustrated in various flowchart diagrams as illustrated in the figures by various elements, as may be used in implementing an exemplary embodiment of the present invention. FIG. 10 depicts an exemplary embodiment of a computer system that may be used in computing devices including those shown and not shown, such as, e.g., but not limited to, service provider computing devices, AI/ML processing devices, cloud-data storage, cloud processing devices, data repository processing and/or storage computing devices, network devices, communication devices, authentication devices, notification devices, requesting devices, response devices, encryption/decryption cryptological processing devices, computing devices, payment processing related devices, network monitoring and analysis devices of example operations illustrated example environment 600, computing devices 1000, devices, servers, application servers, web servers, other wearable and/or mobile devices, sensor capture devices, routers, gateways, data network communication equipment, etc., according to an exemplary embodiment of the present invention. FIG. 10 depicts an exemplary embodiment of a computer system that may be used as client device, a server device (not shown), a network component, router, packet monitor/analyzer, server device, storage, and/or cloud based storage device, application servers, and/or web servers, etc. The present invention (or any part(s) or function(s) thereof) may be implemented using hardware, software, firmware, or a combination thereof and may be implemented in one or more computer systems or other processing systems. In fact, in one exemplary embodiment, the invention may be directed toward one or more computer systems capable of carrying out the functionality described herein. An example of a computer system 1000 is shown in FIG. 10, depicting an exemplary embodiment of a block diagram of an exemplary computer system useful for implementing the present invention. Specifically, FIG. 10 illustrates an example computer 1000, which in an exemplary embodiment may be, e.g., (but not limited to) a personal computer (PC) system running an operating system such as, e.g., (but not limited to) WINDOWS MOBILE™ for POCKET PC, or MICROSOFT® WINDOWS® NT/98/2000/XP/CE/7/8/10/11, etc. available from MICROSOFT® Corporation of Redmond, Wash, U.S.A., SOLARIS® from SUN® Microsystems of Santa Clara, Calif, U.S.A, OS/2 from IBM® Corporation of Armonk, N.Y, U.S.A, Mac/OS, Mac OSX, iOS, from APPLE® Corporation of Cupertino, Calif, U.S.A, etc., or any of various versions of UNIX® (a trademark of the Open Group of San Francisco, Calif, USA) including, e.g., LINUX®, UBUNTU, HPUX®, IBM AIX®, and SCO/UNIX®, etc., ANDROID, Android OS is available from Google, JAVA from Oracle, etc. However, the invention may not be limited to these platforms. Instead, the invention may be implemented on any appropriate computer system running any appropriate operating system. In one exemplary embodiment, the present invention may be implemented on a computer system operating as discussed herein. An exemplary computer system, computer 1000 is shown in FIG. 10. Other components of the invention, such as, e.g., (but not limited to) a computing device, a communications device, a telephone, a personal digital assistant (PDA), a personal computer (PC), a handheld PC, client workstations, thin clients, thick clients, proxy servers, network communication servers, remote access devices, client computers, server computers, routers, web servers, data, media, audio, video, telephony or streaming technology servers, a tablet, a phone, a mobile phone, a cellular phone, a communications device, an iPhone, iOS, a smartphone, an iPad, a tablet based device, an ANDROID OS device, an iOS device, a Symbian based device, a Windows 8, 10, 11, etc., device, etc., may also be implemented using a computer such as that shown in FIG. 10.

The computer system 1000 may include one or more processors, such as, e.g., but not limited to, processor(s) 1004, which may include microprocessors, coprocessors, nanoprocessors, microcontrollers, systems on a chip (SOC), multi-processor systems, parallel processors, CISC type processors, RISC type processors, POWER type processors, ARM-architecture processors, massively parallel processor, graphic processors (GPUs) 1032, cryptographic processors such as, e.g., but not limited to, encryption/decryption processor 1036, quantum computers, etc. The processor(s) 1004 may be connected to a communication infrastructure 1006 (e.g., but not limited to, a communications bus, cross-over bar, or network, etc.). Various exemplary software embodiments may be described in terms of this exemplary computer system. After reading this description, it will become apparent to a person skilled in the relevant art(s) how to implement the invention using other computer systems and/or architectures.

Computer system 1000 may include a display interface 1002 that may forward, e.g., but not limited to, graphics, text, and other data, etc., from the communication infrastructure 1006 (or from a frame buffer, etc., not shown) for display on the display unit 1030, and/or GPU 1032, and/or touchscreen 1034, and/or other input or output, and/or input and output device, sensor-based device, etc.

The computer system 1000 may also include, e.g., but may not be limited to, a main memory 1008, random access memory (RAM), and a secondary memory 1010, etc. The secondary memory 1010 may include, for example, (but not limited to) a hard disk drive 1012 and/or a removable storage drive 1014, representing a floppy diskette drive, a magnetic tape drive, an optical disk drive, a compact disk drive CD-ROM, DVD, Personal Cloud storage, redundant array of inexpensive disks (RAID) array, etc. The removable storage drive 1014 may, e.g., but not limited to, read from and/or write to a removable storage unit 1018 in a well-known manner. Removable storage unit 1018, also called a program storage device or a computer program product, may represent, e.g., but not limited to, a floppy disk, magnetic tape, optical disk, compact disk, etc. which may be read from and written to by removable storage drive 1014. As will be appreciated, the removable storage unit 1018 may include a computer usable storage medium having stored therein computer software and/or data.

In alternative exemplary embodiments, secondary memory 1010 may include other similar devices for allowing computer programs or other instructions to be loaded into computer system 1000. Such devices may include, for example, but not limited to, a removable storage unit 1022 and an interface 1020. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, FLASH memory, application specific integrated circuits (ASICs), field programmable gate arrays (FPGAs), and/or other removable storage units 1022 and interfaces 1020, which may allow software and/or data to be transferred from the removable storage unit 1022 to computer system 1000.

The computing device 1000 may also include a cloud-accessible or cloud-based processing and/or storage solution as may be available from Amazon Web Services available from Amazon of Seattle, WA USA, or Azure cloud available from Microsoft Corporation of Redmond, WA USA, or Google Cloud Service available from Google of Alphabet Corporation, Mountain View, CA USA, among many other network and software communications offerings available from IBM Corporation, Oracle Corporation, and others.

Computer 1000 may also include an input device such as, e.g., (but not limited to) a mouse or other pointing device such as a digitizer, touch-based sensor, and/or a keyboard and/or other data entry device (none of which are labeled).

Computer 1000 may also include output devices, such as, e.g., (but not limited to) display 1030, and display interface 1002. Computer 1000 may include input/output (I/O) devices such as, e.g., (but not limited to) communications interface 1024, cable 1028 and communications path 1026, etc. These devices may include, e.g., but not limited to, a network interface card, and modems (neither are labeled). Communications interface 1024 may allow software and data to be transferred between computer system 1000 and external devices. Examples of communications interface 1024 may include, e.g., but may not be limited to, a modem, a network interface (such as, e.g., an Ethernet card), a communications port, a Personal Computer Memory Card International Association (PCMCIA) slot and card, etc. Software and data transferred via communications interface 1024 may be in the form of signals 1028 which may be electronic, electromagnetic, optical or other signals capable of being received by communications interface 1024. These signals 1028 may be provided to communications interface 1024 via, e.g., but not limited to, a communications path 1026 (e.g., but not limited to, a channel). This channel 1026 may carry signals 1028, which may include, e.g., but not limited to, propagated signals, which may be stored in nontransitory form, and may be implemented using, e.g., but not limited to, wire or cable, local and/or wide area network (LAN/WAN) protocols, Ethernet, Token Ring, FDDI, carried over and y of various physical media, fiber optics, a telephone line, twisted pair, shielded twisted pair, a cellular link, a radio frequency (RF) link, wireless communications, spread spectrum, orthogonal frequency division multiplexing (OFDM), and/or other communications channels, etc.

In this document, the terms “computer program medium” and “computer readable medium” may be used to generally refer to media such as, e.g., but not limited to removable storage drive 1014, a hard disk installed in hard disk drive 1012, and signals 1028, etc. These computer program products may provide software to computer system 1000. The invention may be directed to such computer program products.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct or indirect physical or electrical contact with each other. “Coupled” may mean that two or more elements are in direct physical or electrical contact. However, “coupled” may also mean that two or more elements are not in direct contact with each other, but yet still co-operate or interact with each other.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments of the present invention may include apparatuses for performing the operations herein. An apparatus may be specially constructed for the desired purposes, or it may comprise a general purpose device modified as set forth herein to perform the processing as described to be selectively activated or reconfigured by a software program stored in the device to become a special purpose device capable of performing the subsystem's or submodule's performance functionality and computer and communications systems instructions, and/or by hardware processing such as, e.g., but not limited to, performing certain trusted platform system processing, including exemplary key based encryption/decryption, network monitoring, packet inspection and the like, according to exemplary embodiments.

Embodiments of the invention may be implemented in one or a combination of hardware, firmware, and software. Embodiments of the invention may also be implemented as instructions stored on a machine-readable medium, which may be read and executed by a computing platform to perform the operations described herein. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other form of propagated signals (e.g., carrier waves, infrared signals, digital signals, etc.) when nontransitory, and others.

Computer programs (also called computer control logic), may include object-oriented computer programs, and may be stored in main memory 1008 and/or the secondary memory 1010 and/or removable storage units 1014, also called computer program products. Such computer programs, when executed, may enable the computer system 1000 to perform the features of the present invention as discussed herein. In particular, the computer programs, when executed, may enable the processor 1004 to provide a method to resolve conflicts during data synchronization according to an exemplary embodiment of the present invention. Accordingly, such computer programs may represent controllers of the computer system 1000.

Various artificial intelligence based analysis techniques may be used herein including neural networks, machine learning, any of various well-known AI and ML techniques and processes (e.g., reinforcement learning, dynamic programming, state action reward state action (SARSA), q learning, supervised learning, unsupervised learning, large language models (LLMs), natural language search, meta-learning and interactive request and response, neural networks, convolutional neural networks, statistical heuristics, topic identification and classification, linguistics and semantic processing, tensorflow and openAI libraries, cloud computing services, specific APIs, Microsoft cognitive services, Google cloud AI, Watson AI, offerings from Amazon, Facebook, Baidu, Apple, and others, etc.), and output of such algorithms may be analyzed further as set forth herein to obtain feature vectors and other data which may be used to provide further guidance to users, and/or be integrated for further processing and analysis, authentication, access control, and/or encryption/decryption processing, and coupled via decision support systems, executive information systems, and other graphical user interface enabled network and cyber security monitoring and threat analysis management and processing.

In another exemplary embodiment, the invention may be directed to a computer program product may include a computer readable medium having control logic (computer software) stored therein. The control logic, when executed by the processor 1004, may cause the processor 1004 to perform the functions of the invention as described herein. In another exemplary embodiment where the invention may be implemented using software, the software may be stored in a computer program product and loaded into computer system 1000 using, e.g., but not limited to, removable storage drive 1014, hard drive 1012 or communications interface 1024, etc. The control logic (software), when executed by the processor 1004, may cause the processor 1004 to perform the functions of the invention as described herein. The computer software may run as a standalone software application program running atop an operating system or may be integrated into the operating system.

In yet another embodiment, the invention may be implemented primarily in hardware using, for example, but not limited to, hardware components such as application specific integrated circuits (ASICs), or one or more state machines, etc. Implementation of the hardware state machine so as to perform the functions described herein will be apparent to persons skilled in the relevant art(s).

In another exemplary embodiment, the invention may be implemented primarily in firmware.

In yet another exemplary embodiment, the invention may be implemented using a combination of any of, e.g., but not limited to, hardware, firmware, and software, etc.

Exemplary embodiments of the invention may also be implemented as instructions stored on a machine-readable medium, which may be read and executed by a computing platform to perform the operations described herein. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other form of propagated signals (e.g., carrier waves, infrared signals, digital signals, etc.), and others.

According to an exemplary embodiment, the application system can include an electronic decision support system (DSS) (not shown), policy-based trust platform systems, which can interact, e.g., but not limited to, with computer database management system (DBMS) 1007, and/or electronic interactive, graphical user interface (GUI) system. Each of the exemplary DSS, DBMS and/or EIGUI system, can then, using e.g., but not limited to, a cryptographic processor and/or a crypto chip controller processor 1036, or the like, can then encrypt the data using electronic encryptor, which can make use of one or more cryptographic algorithm electronic logic, which can include encryption code, a cryptographic combiner, etc., and may be stored in encrypted form, according to an exemplary embodiment, in a computer database storage facility, from computer database storage device, and from there the process can continue with use of the cryptographic algorithm electronic logic, and electronic decryptor, which can decrypt and/or provide a process for decrypting encrypted data, and/or by providing such data to the DSS, the DBMS, or the EIGUI, if authorized. By using encryption/decryption, certain algorithms can be used, as described herein, including, e.g., but not limited to, checksum, AES encryption, RSA, PKI, TLS, FTPS, SFTP, etc. and/or other cryptographic algorithms and/or protocols, according to exemplary embodiments.

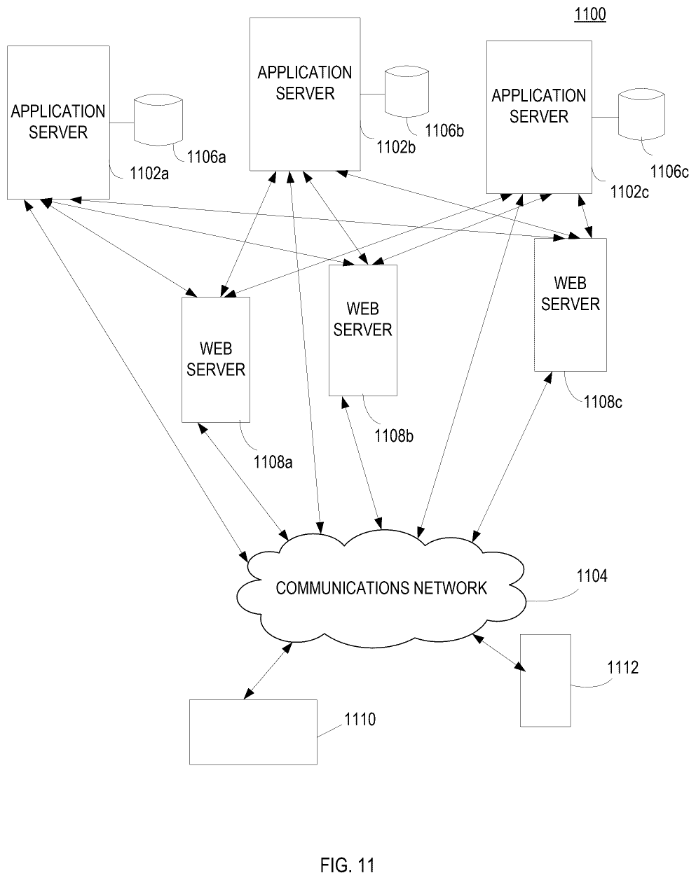

FIG. 11 depicts an example embodiment of an example schematic diagram 1100 of an example illustration of an example computer load balancing example system architecture block diagram of various example system components of any example networked system of various example electronic computing devices of an example system, according to one example embodiment of the present invention.

FIG. 11 depicts an example system network architecture diagram of an example web server and application server embodiment, according to an example embodiment depicts an example embodiment illustrating an example system level hardware architecture 1100 for an example application server, web server embodiment of an example service provider system for providing an example software architecture of various example subsystem components of an example networking system, which may be used to implement various illustrated computational processing including AI/ML processing, in a networked environment, according to one example embodiment, discussed further below.

FIG. 11 depicts an example embodiment of example schematic views 1100 of an example redundant, example communications network environment for providing example load balancing and electronic coupling between example client and server devices in peer-to-peer, client/server, and/or other network topologies as will be well known to those skilled in the relevant art, and may include various example application servers, and file storage devices, web servers, and other networked devices as shown, according to one example embodiment of the present invention, according to an example embodiment of the present invention.

FIG. 11 depicts an example view 1100 of an example embodiment of an exemplary hardware environment architecture block diagram of an example embodiment of an example communication network, which may be coupled to a plurality of exemplary systems of exemplary communication/computing devices, etc., as well as other exemplary client and/or server devices, which may include, e.g., but not limited to exemplary database management systems (DBMS), which may organize and store such application code instructions, and/or data, and may enable other applications such as, e.g., but not limited to, electronic messaging, notifications, analysis, statistical analysis, time entry, billing, payment processing, electronic time tracking, electronic location tracking and determining a worker user location at user clock in selection for time keeping/tracking, electronic reporting of jobs, electronic applications and approvals of jobs, and job detail, and time tracking features, alerting notifications, management, IT management, network management, security and data backup, human resource management, independent contractor tracking, access to, opt in, to terms and conditions, user terms of use, privacy policy, end user license agreement (EULA) access, mobile application EULA access, etc., marketing and affinity management, safety training, safety and employment duties, training, questions and answers, recording, historical access tracking, logging, load balancing, web serving, SAAS access, example cloud-based hosting, application server, and/or browser based application access, etc., for delivering an exemplary interactive HR management SaaS application and example GETGANAS mobile application system, method and/or computer program product, according to one example embodiment of the present invention.

FIG. 11 depicts an exemplary embodiment of an exemplary block diagram 1100 of an example client server reporting networked communications system of various exemplary users of the exemplary interactive HR management and electronic timekeeping application system, method and/or computer program product, according to an exemplary embodiment of the present invention.

FIG. 11 depicts an example system network architecture diagram of an example web server and application server embodiment, according to an example embodiment depicts an example embodiment illustrating an example system level hardware architecture 1100 for an example application server, web server embodiment of an example service provider system for providing an example software architecture of various example subsystem components of an example networking system, which may be used to implement various illustrated computational processing including AI/ML processing, in a networked environment, according to one example embodiment, discussed further below.

FIG. 11 depicts an exemplary schematic block diagram 1100 illustrating an exemplary communications network device hardware architecture 1100 showing exemplary network devices including exemplary application server devices 1102, exemplary storage devices 1106, web server devices 1108, all coupled to an exemplary communications network 1104, which may be coupled to other devices such as, e.g., but not limited to, an artificial intelligence/machine learning processing device, a cyber security environment device, a network security operations environment device, etc., or other devices 1104, user client devices, patient devices, physician devices, diagnostic devices, training devices, user interaction devices, task outputting application server devices, and other user, end user and other internal and/or external user computing devices, smartphone devices, tablet devices, mobile devices, and/or communications devices 1110, which may be coupled to the network communications environment 1104 by one or more physical devices not shown, such as, e.g., but not limited to, network interface cards (NICs), wireless local area network WLAN or wireless fidelity (WI-FI) (IEEE 802.11 communication technologies), physical wires, twisted pair, coaxial cable, fiber optic cable, and/or other communications network cabling, wireless communication access points and/or wireless antennae, wires, wireless media, routers, gateways, access points, exchange carrier facilities, modulator/demodulator (modems), modulating equipment, cellular network exchange devices, etc. Block diagram 1100 may serve as an example logical diagram of associated physical devices. Physical devices may communicate via one or more communications network software protocols via exemplary protocol languages or stacks which may correlate to the Open Systems Interconnect (OSI) stack of communication layers, such as, e.g., but not limited to, Internet Protocol (IP), transmission control protocol (TCP), among various other protocols including, e.g., but not limited to, TCP/IP protocols, UDP, hypertext transfer protocol (HTTP), file transfer protocol (FTP), security protocols (e.g., HTTPS, SFTP, FTPS, TLS, etc.), as will be apparent to those skilled in the relevant art. Various load sharing devices and port sharing devices may be used, as well as protocol tracing and packet tracing devices, which may be able to access such communications data and perform exemplary processing including packet inspection, deep packet inspection, network tracing, packet inspection (e.g., source and destination address, etc.) and the like. Such systems may be used to perform various block chain related distributed ledger technologies so as to replicate data which may be encrypted and decrypted using various algorithms. Certain algorithms may be used which may be resilient to conventional decryption techniques, resilient to brute force attack decryption techniques, and/or may be resilient to quantum computing based decryption techniques. Such software and hardware network analysis and/or processing tools may be used to process communications network traffic internal to, and/or external from an exemplary entity organization and an exemplary network operations center (NOC) network monitoring organization may be able to analyze such network traffic to, e.g., process different types of traffic, image processing, video processing, AI/ML processing, using various sophisticated network operations tools and techniques as well as to perform various automated processing as described further herein.

Various artificial intelligence based analysis techniques may be used herein including neural networks, machine learning, any of various well-known AI and ML techniques and processes (e.g., reinforcement learning, dynamic programming, meta-learning, state action reward state action (SARSA), q learning, supervised learning, unsupervised learning, tensorflow and openAI libraries, cloud computing services, specific APIs, Microsoft cognitive services, Google cloud AI, Watson AI, Large Language Models (LLMs), OpenAI, ChatGPT, offerings from Microsoft, Bing, Google, Bard, Amazon, Facebook, Baidu, Apple, and others, etc.), and output of such algorithms may be analyzed further as set forth herein to obtain feature vectors and other data which may be used to provide further guidance to users via diagnostic tools, diagnostic support tools, physician's assistant and physicians support systems, electronic medical records, decision support systems, executive information systems, and other graphical user interface enabled automation systems, physician and/or provider support computer systems, network monitoring, cyber security monitoring, health management IT support systems, and processing.

### Example Machine Learning Algorithms

Various example embodiments may use artificial intelligence and machine learning techniques. According to one example embodiment, a scikit-learn, open source machine learning library for the Python programming language, may be used to develop a classification supervised machine learning algorithm. Example algorithms may include Random Forest, k-Nearest Neighbors, Convolutional Neural Networks, Quadratic Discriminant Analysis, Logistic Regression, Gradient Boosted Decision Trees, Ada Boost, Naïve Bayes Classifiers and Support Vector Machines, and various other techniques such as, e.g., but not limited to, a supervised learning method, an unsupervised learning method, or a reinforcement learning method, linear regression, logistic regression, decision tree, support vector machine (SVM), naive bayes, k-nearest neighbors (kNN), k-means clustering, random forest, dimensionality reduction, or gradient boosting algorithm.

### Convolutional Neural Networks

Neural networks are a subset of machine learning, at the heart of deep learning algorithms. NNs are comprised of node layers, containing an input layer, one or more hidden layers, and an output layer. Each node connects to another and has an associated weight and threshold. If the output of any individual node is above the specified threshold value, that node is activated, sending data to the next layer of the network. Otherwise, no data is passed along to the next layer of the network.

While feedforward networks are described generally herein, there are various types of neural nets, which are used for different use cases and data types, and may also be used in alternative embodiments. For example, recurrent neural networks are commonly used for natural language processing and speech recognition, whereas convolutional neural networks (also known as ConvNets or CNNs) are more often utilized for classification and computer vision tasks. Prior to CNNs, manual, time-consuming feature extraction methods were used to identify objects in images. However, convolutional neural networks may provide a more scalable approach to image classification and object recognition tasks, leveraging principles from linear algebra, as discussed herein, e.g., matrix multiplication, to identify patterns within an image or frame of a video. CNNs can be computationally demanding, requiring graphical processing units (GPUs) to train models, among other high performance processing solutions, such as cloud-based processing and the like.

Fukushima and LeCun performed early research around convolutional neural networks and published Backpropagation Applied to Handwritten Zip Code Recognition in the 1980s, successfully applying backpropagation to train neural networks to identify and recognize patterns within a series of handwritten zip codes. LeNet-5, in the 1990s applied the research to document recognition. Since then, various CNN architectures have emerged with the new datasets, such as MNIST and CIFAR-10, and competitions, like ImageNet Large Scale Visual Recognition Challenge (ILSVRC). Some of these other architectures include: AlexNet; VGGNet; GoogLeNet; ResNet; ZFNet, and LeNet-5.

An example embodiment of an example software architecture block diagram of an example software architecture may include, e.g., an example hardware layer, an example operating system layer, an optional example trusted computer isolated area for handling example encryption/decryption key handling for user access to the example application, example software application layer modules which may include, e.g., but not limited to, an example biometric and/or personal health record authorization access information, sensed data, data history, health metric indicator ranges, optimal, normal, severity, units, and other patient and physician information record data, indicator detail data, history data, medicine data, medical diagnosis, medication, indicator metric fields, view information, historical database, memory storage database, an user profile database of user profile records, verification information, identification information, patient information, patient health record information, electronic health record data, medical testing results data, patient data, patient scheduling data, physician data, patient demographic and/or health related data, email communication, training, audio video content, history over time, logging changes, notifications, communications, training, data entry, search fields, indicator search query, indicator import/export/enable/disable, etc., according to example embodiments of the present invention.

Various example embodiments of example database architectures as may be used to store user data, as well as exemplary interactive spherical membrane visualization display graphical user interface related data, according to an exemplary embodiment of the present invention, may include example hierarchical taxonomy, and/or an ontology, as may be used in certain example embodiments, according to various example embodiments of the present invention.

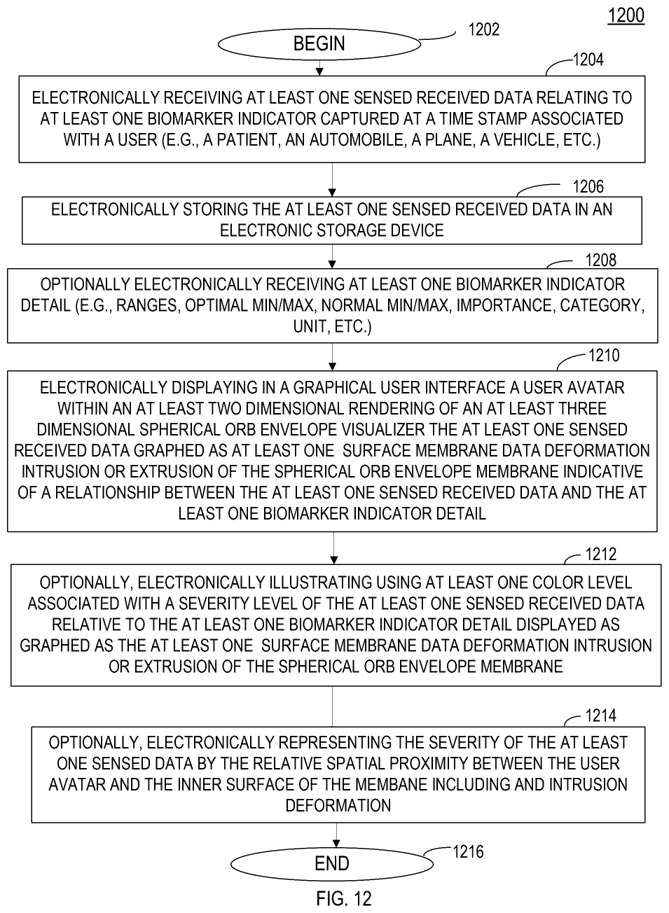

FIG. 12 depicts an example embodiment of an example flow chart diagram 1200 illustrating an example high level flow chart of example process flow of an example display visualizer user interface apparatus, method, system and computer program product, according to one example embodiment of the present invention.

FIG. 12 depicts an example embodiment of an example flow diagram 1200 of an example process of the example user interacting with the example GUI application 100 on an example device as it may interact with either locally stored data and/or to remote stored data, which may be on the backend accessible via an example network with the example SAAS application server, according to one example embodiment of the present invention.

Flow diagram 1200 may begin with 1202 and may continue immediately to 1204, according to one example embodiment.

In 1204, according to one example embodiment, flow diagram 1200 in an example embodiment may electronically receive at least one sensed received data relating to at least one biomarker indicator captured at a time stamp associated with a user (e.g., a patient, an automobile, a plane, a vehicle, etc.) and from 1204, flow diagram 1200 may continue with 1206, according to one example embodiment.

In 1206, according to one example embodiment, flow diagram 1200 may electronically store the at least one sensed received data in an electronic storage device and from 1206, flow diagram 1200 may continue with 1208, according to an example embodiment.

In 2208, according to one example embodiment, flow diagram 1200 may electronically optionally, receive at least one example biomarker indicator detail (e.g., range information such as, e.g., but not limited to, optimal min/max, normal min/max, importance, category, unit, etc.) and from 1208, flow diagram 1200 may continue with 1210, according to an example embodiment.

In 1210, according to one example embodiment, flow diagram 1200 may electronically display in an example graphical user interface a user avatar within an at least two dimensional rendering of an at least three dimensional spherical orb envelope visualizer the at least one sensed received data graphed as at least one surface membrane data deformation intrusion or extrusion of the spherical orb envelope membrane indicative of a relationship between the at least one sensed received data and the at least one biomarker indicator detail, and from 1210, flow diagram 1200 may continue with 1212, according to an example embodiment.

In 1212, according to one example embodiment, flow diagram 1200 may electronically optionally, illustrate using at least one color level associated with a severity level of the at least one sensed received data relative to the at least one biomarker indicator detail displayed as graphed as the at least one surface membrane data deformation intrusion or extrusion of the spherical orb envelope membrane, and from 1212, flow diagram 1200 may continue with 2214, according to an example embodiment.

In 1214, according to one example embodiment, flow diagram 1200 may, e.g., optionally, electronically represent the severity of the at least one sensed data by the relative spatial proximity between the user avatar and the inner surface of the membrane including and intrusion deformation, and from 1214, flow diagram 1200 may continue with 1216, according to an example embodiment.

In 1216, according to one example embodiment, flow diagram 1200 may immediately end.

According to some example embodiments, example modules may be set up as state diagram state machines, in some example embodiments, and may include, e.g., but not limited to, example cloud storage, and/or access, cloud instances by customer, enhanced AI processing including example LLM generated natural language interactive chatbots and/or robotic process automation, use of electronic state information files such as, e.g., but not limited to, e.g., a cookie and/or other automated database access tools such as, e.g., but not limited to, query tools, queries, a database management systems, example relational database management structured query language (SQL) queries, natural language queries, other database types, hierarchical databases, noSQL database, distributed ledge blockchain, public or private block chain, tokenized nonfungible token (NFT), finite state machines (FSM), and/or natural language interaction AI/ML/CNN/LLM, machine learning and natural language processing and search queries, autocompletion of certain information, multilingual language translation features, native language communications, enhanced distributed database distributed ledger block chain enhancements for tracking of electronic smart contracts and/or payment processing, encryption/decryption and use of public/private key encryption techniques to access and/or update data records, etc., according to example embodiments.

The visualizer GUI software application may be web browser accessible, an applet, a software application, a SAAS application server backend system accessible application, a client server application, a peer to peer application system, a local, remote, and/or hybrid system which may facilitate a user visualizing using example embodiments of the visualizer application GUI, various complex multi-dimensional data about an example entity, such as, e.g., but not limited to, an example medical patient of an example physician, enabling providing an intuitive visualization displaying an understandable example spherical (and/or other three dimensional volume shaped) orb membrane envelope user interface element showing particular indicators which may be in close proximity to the illustrated avatar are of significant severity and may allow visualizing change in these various metrics which may relate, e.g., but not limited to, a patient's health and/or potential longevity with respect to the patient's medical health and likelihood of aging gracefully, according to one example embodiment, and highlighting potential problem areas to focus on, impacts of medications, certain biomarkers, etc., and potential medical interventions and/or other patient interventions to reverse negative indications displayed, providing display of various example detailed recommendations and/or diagnoses, and/or predictions of potential outcomes, and may enable holding physician/patient communications and/or interactions to constructively improve patient health, according to one example embodiment.

According to one example embodiment, the example visualizer may be used to provide an intuitive medical visualization illustrating the health of the patient by the relative space surrounding an example avatar within an example membrane volumetric orb, which may be a spherical orb and/or other continuous shape such as an egg shaped orb, an oval and/or ellipse cross-section orb, and/or other shape, and an avatar may be in certain embodiments gender selectable so an example female avatar 130a of FIG. 4B and/or an example male avatar 130 may be provided, according to example embodiments. According to other example embodiments, a health of a person may be visualized. According to other example embodiments, a likelihood of a person dying may be visualized. According to other example embodiments, an illustration of likely factors impinging on the longevity of a person may be visualized.

According to other example embodiments, a health of an example pet may be visualized. For example, a veterinarian may display for a pet owner a visualization of various indicators affecting the health of a pet, from e.g., but not limited to, health indicators, and/or age related factors, and/or other medical test data, etc., according to one example embodiment.

The visualizer may be used for other example applications for visualizing other complex multi-dimensional data, such as, e.g., but not limited to, providing an electronic graphical UI display of a visualization of various maintenance tasks and/or recommended tasks for an example vehicle such as, e.g., but not limited to, an automobile, car, truck, bus, boat, aircraft and/or other vehicle. According to one example embodiment, for an example automobile maintenance status visualization (e.g., replacing tires on a car, changing timing belt, performing lube, oil change, filter replacement, etc., illustrating aging of subsystems of the vehicle, e.g., need for tire rotations, brake replacements, tuneups, replacement of certain fluids, brake fluids, washer fluids, etc.), certain higher priority indicators could be shown spatially in the proximity of the envelope to an avatar, which could include an image of the car, for example, according to one example embodiment.

According to another embodiment, similarly maintenance information for an aircraft could be displayed and enable a visual representation of sensed multidimensional data, showing visually intuitive prioritization of higher severity indications such as the need for deicing of an aircraft in cold weather, replacement of landing gear based on tracked usage and/or sensed wear, maintenance and/or other subsystem repair, maintenance and/or replacement, with an example intuitive illustration of the spherical membrane surrounding an example avatar illustration of the aircraft, in certain example embodiments the spherical orb could be alternatively replaced with an example oval elliptical volume membrane to accommodate an example lengthy plane avatar illustration and/or example automobile avatar of other example embodiments, according to other embodiments.

According to another example embodiment, the GUI visualizer may assist in displaying a visualization a state of a relationship between, and/or marriage of two persons, using multiple avatars in another example orb GUI, where indicators of data relevant to the relationship of two persons and the relative health of the relationship may be represented by the proximity of intrusions from an encapsulating membrane, according to an example embodiment.

### Example Embodiment of Social Sharing Feature

According to one example embodiment, an example social sharing function may be included with an example embodiment of the example membrane envelope visualizer software application. According to one example embodiment of an example user orb membrane visualizer, example MORRBs, as shown on menu element 704 of menu 702 in screencapture 700 of FIG. 7. According to one example embodiment, the example social share feature 704 may support various sharing such as, e.g., but not limited to, sharing of screen captures of one's MORRB, and/or social sharing interactive access to one's MORRB software application, and/or sharing at least a portion of or up to an entirety of, or any user-selectable subset of, e.g., but not limited to visualizer information, sharing portions to gamify and/or create example competitions, according to one example embodiment. In one competition example embodiment one may share in order to facilitate conducting, e.g., but not limited to, “group health competitions,” “group support sessions,” “health matching for dating,” HIPAA based sharing of health privacy information with authorized users such as, e.g., but not limited to, physicians, care providers, family, relatives, etc., according to one example embodiment. Essentially, according to one example embodiment, an example feature may include, e.g., but not limited to, that an example sharing function may (with permission granted by the owner of the example health information, using, e.g., access control registration, such as granting access to a user name/email, as well as e.g., password, biometric, and/or other authentication/verification, access controls, etc.) may allow a given person to securely share that given person's example MORRBs, and/or at least a portion of that given person's MORRB, with at least one other user, and/or to even post a portion or up to the entirety of the given person's MORRB to a social media hosted site, so that one could initiate and/or facilitate and/or participate in an example Health competition such as, e.g., but not limited to, a Health Olympics Competition, or the like, where, according to one example embodiment, any user, a group of users, and/or every user participating in the example competition, can view a display, and/or access, and/or interactively access, MORRB related data and/or the software application and the user to which the information/data is shared may view the displayed visualization showing how well the user is doing relative to, and/or in comparison to others, according to one example embodiment. According to one example embodiment, AI/ML may analyze a shared performance and may assess potential predictive outcomes, and/or recommend certain changes in activity by the user from whom the information was shared, according to one example embodiment. The bigger the example MORRB bubble and/or or membrane orb, according to one example embodiment., and the more green excursions membrane deformations shown, reaching out from the bubble, based on the user's captured and/or sensed indicator data, the better the given user is doing at improving the user's health outcomes, according to one example embodiment. Example viral spreading of access to, and/or knowledge of the example membrane orb envelope application may be achieved by having users share using the example sharing feature, by indirectly sharing the use of the example application with the user's example social media contacts, according to one example embodiment. Sharing, according to one example embodiment, may be shared via social media network and/or other electronic means such as, e.g., but not limited to, sharing an example social media post, sharing an example electronic email, sharing an example SMS message, sharing an example MMS message, sharing an example Internet notification and/or electronic communication, sharing an example access right to access controls to have access to an example user's data, such as, e.g., but not limited to, sharing to an example service provider, sharing to an example physician, sharing to an example provider, sharing to an example health care provider, sharing to an example insurance company to seek a discount on health insurance, sharing to an example new owner for an example asset and/or entity related data or information, etc., according to one example embodiment.

### Example Embodiment of Digital Me Feature

According to another example embodiment, an example digital me—“DigiMe” feature function may be included with an example embodiment of the example membrane envelope visualizer software application, according to one example embodiment. According to one example embodiment, a user may select from one or more, example gender specific example avatars, such as, e.g., but not limited to, an example three dimensional human body shaped figure, which may appear in an example 3d chrome and/or silver and/or gray appearance male avatar and/or female avatar, which is placed disposed within the, e.g., but not limited to, example middle and/or midst of the example spherical orb membrane referred to generally herein as the “MORRB,” according to one example embodiment. According to one example embodiment, an example membrane envelope visualizer software application may provide an example enhanced version of the example avatar, such as, e.g., but not limited to, an example customized and/or customizable (by user and/or by AI) avatar representation, according to an example embodiment. An example man/woman/child/elder avatar in the middle may be enhanced, according to one example embodiment, by being modified to become an advanced almost supernaturally AI digital version of the represented user (i.e., e.g., but not limited to, a person, a relative, a parent, a child, children, an aged relative in care, an example customized avatar, which may include, e.g., but not limited to, an example animated, caricatured, and/or realistically simulated, scanned, and/or customized by example filter, example menu based prompts, example AI/ML sensing and/or user selection, and/or use of an example electronic avatar from a third party, etc.) such that the example enhanced avatar may be thought to “know” you (via, e.g., but not limited to, reading (i.e., electronically receiving, analyzing, and/or processing and/or predicting example simulated appearances and/or receiving everything the associated user has ever written, and/or any data captured about the person including, e.g., but not limited, any still images, video, audio, voice recognition, speech recognition, videocapture, audio capture, and/or stored digital scans, captured three dimensional volumetric scans of surface imagery, and/or 3D renderings from example multiple two dimensional images and/or 3D realistic scans from e.g., but not limited to, RENDERPEOPLE.COM, ARTEC3D.COM, ARTEC SHAPIFY, a 3D body scanner, OPENSCAN, ARTIST 3D, captured from handheld scanners, photogrammetry processing software, 3D models, and/or RENDERPEOPLE models, free models, posed people models, rigged people models, animated people models, 4D people models, motion models, location hosted models (e.g., at a beach setting, in a professional setting, in a hospital setting, in a different weather setting, a seasonal clothed setting, in a work setting, in a casual setting, in a setting with other family member, spouse, child, parent, couples, in an athletic setting, casually animated models, groups of models, etc., and from interacting with you, over time, and/or via data capture, and/or predictive modeling based on example demographic information and/or captured user information, and/or customized user information, etc.) and be able to look like a given user and/or converse with a given user, using e.g., but not limited to, example natural language communication such as, e.g., but not limited to, an example chat bot and/or generative pre-trained AI/ML/LLM and/or deep learning model, according to various example embodiments, and/or may encourage the example user to take certain steps going forward, and/or admonish the given user for certain observed/analyzed/determined behaviors and/or changes obtained from electronic analysis of the captured and/or sensed data received by the example system, over time, according to one example embodiment, and/or may include various other example according to one example embodiment. Example embodiment may also be able to display predicted future outcomes, and/or enable a user to access potential what-if scenarios to be viewed, according to one example embodiment. An example embodiment may display a potential future if certain trends continue, for example, but not limited to, “showing” a user physically on the example illustrated avatar illustration/representation of the user what a currently trending set of behaviors may be likely predicted to lead to, in, e.g., but not limited to, example future time periods such as, e.g., but not limited to 6-9 months, 12 months, less, more, greater time periods, lesser time periods, etc. if the example user does not take positive steps and/or if the user continues to take negative steps and/or steps not believed to be negative, such as, e.g., but not limited to, continuing to drink alcohol, increasing a user's weight, inactivity, exercise, taking medicine to address hypertension, not taking medicine to address hypertension, taking medicine, not taking medicine, taking supplements, not taking supplements, changing a diet, not changing a diet, using an intervention, not using an intervention, using sun block, not using sunblock, simulating changes in biological clock, epigenetic clock, using machine learning models and/or electronic aging simulation predictive appearance models, illustrating changes in proteomic, epidemiological data, biological data, life science captured data, biomarker data, sensed data, wearable sensor-based sensed data, light exposure sensed and/or captured data, environmental data, environment climate related data, medical history data, medicinal intervention history data, transcriptomic data, metabolic data, cellular change data, and/etc., according to one example embodiment. According to one example key element, in an example embodiment an example avatar may become a given user's or entity's or asset's alter ego “Man in the Middle,” testable model, where a user and/or other authorized user with authorized access can, e.g., test dietary, and/or behavioral and/or environmental “what if” example scenarios, and/or models and may use electronic digital simulations of predictive, potential changes to example observed data, as example interventions, and/or potential example therapies, example diagnoses, example medicinal interventions, example changes in lifestyle, etc. enabling or facilitating example testing without doing the actual proposed interventions, scenario, etc., to the user's actual body and/or entity, to potentially improve a predicted future outcome based on example historical trends, based on example machine learning/deep learning/neural networks, and/or convolutional neural networks trained with datasets of example patients and/or related outcomes, according to one example embodiment. According to one example embodiment, an example user may also be able to view using example features displaying example interior anatomical effects such as, e.g., but not limited to, example how smoking is making one's lungs dark, and/or at even lower levels, e.g., how the air sacs in a user's lungs may be becoming less permeable, and/or irritated and/or inflamed (which may be predicted to potentially lead to cancer and/or other illness, etc.) and/or, alternatively, if, e.g., a user instead gives up smoking, how long since, impacts of stopping, impacts of duration of quitting, and/or impacts of when the user quit and/or has remained clear of use of tobacco and/or other smoking products, vaping, pot smoking, etc., and/or how the lungs will clear, and example predictions of how and when, based on captured machine learned and/or neural networks of expert system data—to provide the example user, example incentives that the user may view, displayed by the example system in an example simulated, predicted illustration depicting an example predicted change, as shown in the example depiction, according to one example embodiment. In one example embodiment, the avatar may be shown as a male avatar, however, a user may choose a female gender avatar 130a, according to an example embodiment, as shown in FIG. 4B.

While various embodiments have been described above, it should be understood that they have been presented by way of example only, and not limitation. Thus, the breadth and scope of the disclosure should not be limited by any of the above-described exemplary embodiments, but should instead be defined only in accordance with the following claims and their equivalents. It will be apparent to those skilled in the art that various modifications and variations can be made without departing from the spirit or scope of the disclosure. Thus, it is intended that the disclosure cover the modifications and variations provided they come within the scope of the appended claims and their equivalents, and it should be understood, of course, that the foregoing relates to exemplary embodiments of the invention and that modifications may be made without departing from the spirit and scope of the present invention.

What is claimed is:

1. A computer-implemented method of electronically displaying a multi-dimensional complex data visualization in at least one graphical user interface (GUI) on at least one electronic display coupled to at least one electronic computer processor and at least one electronic memory storage device, the method comprising:
a) electronically receiving, by the at least one electronic computer processor, at least one sensed received data relating to at least one indicator;
b) electronically storing, by the at least one computer processor, said at least one sensed received data in the at least one electronic memory storage device;
c) electronically receiving, by the at least one computer processor, at least one indicator detail of said at least one indicator related to said at least one sensed received data; and
d) electronically displaying in the graphical user interface, by the at least one computer processor, at least one avatar within an at least two dimensional rendering of an at least three dimensional orb visualizer said at least one sensed received data graphically displayed as at least one surface membrane comprising at least one data deformation comprising at least one of at least one intrusion data deformation or at least one extrusion data deformation of said at least one surface membrane, wherein an envelope volume is formed between an inner surface of said at least one surface membrane and an outer surface of said at least one avatar, wherein said envelope is indicative of an associated relationship between said at least one sensed received data and said at least one indicator detail.

2. The computer-implemented method according to claim 1, wherein the at least one graphical user interface (GUI) comprises at least one or more of:
an internet browser hypertext markup language (HTML) app;
a messenger app;
a texting app;
an IOS-compatible app;
an ANDROID-compatible app;
a mobile OS-compatible app;
an electronic messaging app;
an electronic widget app;
an hypertext transfer protocol (HTTP) browser-enabled app;
an hypertext markup language (HTML) enabled app;
a web-based application;
a JAVA-compatible app;
a PYTHON script app; or
an interactive graphical user interface app.

3. The computer-implemented method according to claim 1, wherein the at least one graphical user interface (GUI) further comprises at least one or more of:
e) electronically illustrating in the graphical user interface, by the at least one computer processor, using at least one color level displayed in said GUI, associated with a severity level of the at least one sensed received data relative to the at least one indicator detail displayed graphically as said at least one surface membrane comprising at least one intrusion data deformation, or at least one extrusion data deformation of said at least one surface membrane of said at least three dimensional orb visualizer;
f) electronically representing visually by displaying, by the at least one computer processor, a severity of said at least one sensed data by the relative spatial proximity illustrated between the inner surface of said at least one surface membrane and an outer surface of said at least one avatar;
g) electronically enabling zooming in to display, by the at least one computer processor, of said GUI of said at least three dimensional orb visualizer;
h) electronically enabling interactive timeline review display, by the at least one computer processor, of said GUI of said at least three dimensional orb visualizer;
i) electronically enabling interactive temporal increment of historical data display, by the at least one computer processor, of said GUI of said at least three dimensional orb visualizer;
j) electronically enabling interactive filtering of severity indicator information data display, by the at least one computer processor, of said GUI of said at least three dimensional orb visualizer; or
k) electronically enabling note capture and display, by the at least one computer processor, on said GUI of said at least three dimensional orb visualizer.

4. The computer-implemented method according to claim 1, wherein the at least one graphical user interface (GUI) further comprises:
e) electronically illustrating in the graphical user interface, by the at least one computer processor, using at least one color level displayed in said GUI, associated with a severity level of the at least one sensed received data relative to the at least one indicator detail displayed graphically as said at least one surface membrane comprising at least one intrusion data deformation, or at least one extrusion data deformation of said at least one surface membrane of said at least three dimensional orb visualizer;
f) electronically representing visually by displaying, by the at least one computer processor, a severity of said at least one sensed data by the relative spatial proximity illustrated between the inner surface of said at least one surface membrane and an outer surface of said at least one avatar;
g) electronically enabling zooming in to display, by the at least one computer processor, of said GUI of said at least three dimensional orb visualizer;
h) electronically enabling interactive timeline review display, by the at least one computer processor, of said GUI of said at least three dimensional orb visualizer;
i) electronically enabling interactive temporal increment of historical data display, by the at least one computer processor, of said GUI of said at least three dimensional orb visualizer;
j) electronically enabling interactive filtering of severity indicator information data display, by the at least one computer processor, of said GUI of said at least three dimensional orb visualizer; and
k) electronically enabling note capture and display, by the at least one computer processor, on said GUI of said at least three dimensional orb visualizer.

5. The computer-implemented method according to claim 1, wherein said at least one sensed received data was captured at a time stamp and wherein said at least one sensed received data is associated with a user.

6. The computer-implemented method according to claim 1, wherein said user comprises at least one or more of:
a patient user;
an entity;
an asset;
a vehicle;
an automobile;
an aircraft;
a plane;
a boat;
an animal;
a pet;
a dog;
a horse;
a cat; or
a cow.

7. The computer-implemented method according to claim 1, wherein said at least one avatar comprises at least one or more of:
an avatar of a patient user;
an avatar of a female patient user;
an avatar of a male patient user;
a user customizable appearance avatar of a patient user;
a user selectable avatar of a user;
an avatar of an entity;
an avatar of an asset;
an avatar of a vehicle;
an avatar of an automobile;
an avatar of an aircraft;
an avatar of a plane;
an avatar of a boat;
an avatar of an animal;
an avatar of a pet;
an avatar of a dog;
an avatar of a horse;
an avatar of a cat; or
an avatar of a cow.

8. The computer-implemented method according to claim 1, wherein said at least one indicator detail comprises at least one or more of:
at least one biomarker indicator detail;
at least one health test indicator detail;
at least one health metric indicator detail; or
at least one physical health related detail.

9. The computer-implemented method according to claim 1, wherein said at least one indicator detail comprises at least one or more of:
at least one range;
at least one optimal range;
at least one minimum;
at least one maximum;
at least one normal range;
at least one normal minimum;
at least one normal maximum;
at least one importance;
at least one category of said indicator; or
at least one units of said indicator.

10. The computer-implemented method according to claim 1, wherein said at least one indicator detail comprises at least one or more of:
at least one asset maintenance related indicator detail;
at least one asset test indicator detail;
at least one asset maintenance metric indicator detail; or
at least one asset service-related detail.

11. The computer-implemented method according to claim 1, wherein said at least three dimensional orb visualizer comprises at least one or more of:
a spherical orb shape;
an elliptical orb shape;
an amorphous continuous encapsulation shape;
an egg orb shape;
an oval orb shape;
an enclosed orb shape;
a cube orb shape; or
a volumetric orb shape.

12. The computer-implemented method according to claim 1, wherein the at least one graphical user interface (GUI) comprises at least one or more of:
a software as a service (SaaS) cloud-hosted application service provider software application;
a natural language search query tool for interacting with the software application;
a touchscreen pinch/zoom enabled application;
a mouse pointer enable application;
a generative artificial intelligence (AI) pre-trained machine learning (ML) large language model (LLM) natural language query tool for interacting with the software application;
a deep learning convolutional neural network enabled generative artificial intelligence (AI) pre-trained machine learning (ML) large language model (LLM) natural language query tool for interacting with the software application; or
a deep learning convolutional neural network enabled generative artificial intelligence (AI) pre-trained machine learning (ML) large language model (LLM) natural language query tool for interacting with the software application comprising speech recognition and speech synthesis, natural language interaction with the software application.

13. A system of electronically displaying a multi-dimensional complex data visualization in at least one graphical user interface (GUI) application, the system comprising:
at least one electronic computer processor;
at least one electronic touchscreen display input output device or at least one display and at least one mouse pointer device;
at least one electronic memory storage device; and
at least one operating system executing upon said at least one electronic computer processor said electronic computer processor configured to provide the at least one graphical user interface (GUI) application configured to electronically display the multi-dimensional complex data visualization, wherein the at least one electronic computer processor of the system is configured to:
a) electronically receive at least one sensed received data relating to at least one indicator;
b) electronically store said at least one sensed received data in said at least one electronic memory storage device;
c) electronically receive at least one indicator detail of said at least one indicator related to said at least one sensed received data; and
d) electronically display in the at least one graphical user interface at least one avatar within an at least two dimensional rendering of an at least three dimensional orb visualizer said at least one sensed received data graphically displayed as at least one surface membrane comprising at least one data deformation comprising at least one of at least one intrusion data deformation or at least one extrusion data deformation of said at least one surface membrane, wherein an envelope volume is formed between an inner surface of said at least one surface membrane and an outer surface of said at least one avatar, wherein said envelope is indicative of an associated relationship between said at least one sensed received data and said at least one indicator detail.

14. The system according to claim 13, wherein said at least one sensed received data is captured from at least one electronic sensor on at least one or more of:
at least one sensor-based device;
at least one wearable device;
at least one health sensor-based device;
at least one physical health information capture device; or
at least one mobile device comprising at least one sensor.

15. A non-transitory computer program product embodied on a computer accessible storage medium, comprising at least one instruction, which when executed on at least one electronic computer processor performs a method of electronically displaying a multi-dimensional complex data visualization in at least one graphical user interface (GUI) on at least one electronic display coupled to at least one electronic computer processor and at least one electronic memory storage device, the method comprising:
a) electronically receiving, by the at least one electronic computer processor, at least one sensed received data relating to at least one indicator;
b) electronically storing, by the at least one computer processor, said at least one sensed received data in the at least one electronic memory storage device;
c) electronically receiving, by the at least one computer processor, at least one indicator detail of said at least one indicator related to said at least one sensed received data; and
d) electronically displaying in the graphical user interface, by the at least one computer processor, at least one avatar within an at least two dimensional rendering of an at least three dimensional orb visualizer said at least one sensed received data graphically displayed as at least one surface membrane comprising at least one data deformation comprising at least one of at least one intrusion data deformation or at least one extrusion data deformation of said at least one surface membrane, wherein an envelope volume is formed between an inner surface of said at least one surface membrane and an outer surface of said at least one avatar, wherein said envelope is indicative of an associated relationship between said at least one sensed received data and said at least one indicator detail.

16. The method according to claim 1, further comprising at least one or more of:
f) electronically receiving, by the at least one electronic computer processor, a request to share and electronically sharing access to the graphical user interface to an authorized third party via at least one or more of:
electronic social network;
electronic communication;
electronic mail;
electronic message;
electronic social media post; or
electronic access control;
g) electronically gamifying, by the at least one electronic computer processor, receiving a request to enable share to a plurality of users and electronically sharing access to the graphical user interface to said plurality of users, authorized to have access to the GUI via at least one or more of:
electronic social network;
electronic communication;
electronic mail;
electronic message;
electronic social media post; or
electronic access control; or
h) electronically receiving, by the at least one electronic computer processor, a request to share and electronically sharing access to the graphical user interface to an authorized third party wherein the authorized third party comprises least one or more of:
a physician;
a service provider;
a doctor;
an insurance carrier;
a care giver;
a health care provider;
a family member;
a group of users;
a group of competitors in a competition;
an owner of an entity;
an owner of an asset; or
an artificial intelligence/machine learning deep learning analyzer.

17. The method according to claim 1, further comprising at least one or more of:
f) electronically receiving, by the at least one electronic computer processor, a request to electronically customize said avatar and electronically providing user access via the graphical user interface to tools to customize an appearance of said avatar comprising at least one or more of:
electronically selecting an example gender customized appearance of said avatar;
electronically selecting an example realistically featured customized appearance of said avatar;
electronically selecting an example single color appearance of said avatar;
electronically customizing an appearance of said avatar via artificial intelligence and/or machine learning;
electronically customizing an appearance of said avatar via user and/or automated selection of a selection from a library of example avatar models;
electronically customizing an appearance of said avatar comprising at least one or more of:
selecting an approximately similarly aged avatar;
selecting an animated avatar;
selecting a caricatured avatar;
selecting a realistically simulated avatar;
selecting a 3D scanned avatar;
selecting an automatically changed avatar;
selecting a posed avatar;
selecting a seasonally clothed avatar;
selecting a transparent avatar; or
selecting a group of a plurality of avatars; or
g) electronically receiving, by the at least one electronic computer processor, a request to electronically perform a what if scenario, and automatically customizing said avatar and electronically displaying via the graphical user interface said customized appearance of said avatar comprising at least one or more of:
electronically customizing based on performing at least one extrapolation of a trend of past historical data, into a predictive future based on said what if scenario to at least one or more of:
display an improvement in appearance of said avatar if said at least one extrapolation based on said what if scenario is desirable;
display a change in appearance of said avatar;
display an undesirable change in appearance of said avatar if said at least one extrapolation is undesirable;
display a change in appearance of said avatar based on an artificial intelligence or machine learning neural network of expected changes based on said what if scenario;
display a change in appearance of at least a portion of said avatar based on an artificial intelligence or machine learning neural network of expected changes based on said what if scenario; or
display a change in appearance of at least a portion of at least one internal organ of said avatar based on a predicted outcome of said what if scenario.

18. The method according to claim 1, further comprising:
f) electronically receiving, by the at least one electronic computer processor, a request to share and electronically sharing access to the graphical user interface to an authorized third party via at least one or more of:
electronic social network;
electronic communication;
electronic mail;
electronic message;
electronic social media post; or
electronic access control;
g) electronically gamifying, by the at least one electronic computer processor, receiving a request to enable share to a plurality of users and electronically sharing access to the graphical user interface to said plurality of users, authorized to have access to the GUI via at least one or more of:
electronic social network;
electronic communication;
electronic mail;
electronic message;
electronic social media post; or
electronic access control;
h) electronically receiving, by the at least one electronic computer processor, a request to share and electronically sharing access to the graphical user interface to an authorized third party wherein the authorized third party comprises least one or more of:
a physician;
a service provider;
a doctor;
an insurance carrier;
a care giver;
a health care provider;
a family member;
a group of users;
a group of competitors in a competition;
an owner of an entity;
an owner of an asset; or
an artificial intelligence/machine learning deep learning analyzer;
i) electronically receiving, by the at least one electronic computer processor, a request to electronically customize said avatar and electronically providing user access via the graphical user interface to tools to customize an appearance of said avatar comprising at least one or more of:
electronically selecting an example gender customized appearance of said avatar;
electronically selecting an example realistically featured customized appearance of said avatar;
electronically selecting an example single color appearance of said avatar;
electronically customizing an appearance of said avatar via artificial intelligence and/or machine learning;
electronically customizing an appearance of said avatar via user and/or automated selection of a selection from a library of example avatar models;
electronically customizing an appearance of said avatar comprising at least one or more of:
selecting an approximately similarly aged avatar;
selecting an animated avatar;
selecting a caricatured avatar;
selecting a realistically simulated avatar;
selecting a 3D scanned avatar;
selecting an automatically changed avatar;
selecting a posed avatar;
selecting a seasonally clothed avatar;
selecting a transparent avatar; or
selecting a group of a plurality of avatars; and
j) electronically receiving, by the at least one electronic computer processor, a request to electronically perform a what if scenario, and automatically customizing said avatar and electronically displaying via the graphical user interface said customized appearance of said avatar comprising at least one or more of:
electronically customizing based on performing at least one extrapolation of a trend of past historical data, into a predictive future based on said what if scenario to at least one or more of:
display an improvement in appearance of said avatar if said at least one extrapolation based on said what if scenario is desirable;
display a change in appearance of said avatar;
display an undesirable change in appearance of said avatar if said at least one extrapolation is undesirable;
display a change in appearance of said avatar based on an artificial intelligence or machine learning neural network of expected changes based on said what if scenario;
display a change in appearance of at least a portion of said avatar based on an artificial intelligence or machine learning neural network of expected changes based on said what if scenario; or
display a change in appearance of at least a portion of at least one internal organ of said avatar based on a predicted outcome of said what if scenario.

19. The method according to claim 1, wherein said (b) of said electronically storing comprises at least one or more of:
electronically storing said at least one sensed received data in at least one hierarchical database;
electronically storing said at least one sensed received data in at least one relational database;
electronically storing said at least one sensed received data in at least one structured query language (SQL) relational database;
electronically storing said at least one sensed received data in at least one graph database; or
electronically storing said at least one sensed received data in at least one No SQL graph database.

20. The method according to claim 1, wherein said (d) of said electronically displaying in the graphical user database comprises electronically displaying at least one predictive change in appearance to said avatar, based on at least one what if scenario.
